(19) United States
(12) Patent Application Publication
(10) Pub. No.: US 2019/0209671 A1
(43) Pub. Date: Jul. 11, 2019

(54) Specific Chimeric Antigen Receptor T Cells Targeting to CD47, Preparation Method and Application Thereof

(71) Applicant: NANJING KAEDI BIOTECH INC, Nanjing (CN)

(72) Inventors: Hongjiu Dai, Nanjing (CN); Chunjiang Yue, Nanjing (CN)

(73) Assignee: NANJING KAEDI BIOTECH INC, Nanjing (CN)

(21) Appl. No.: 16/285,246

(22) Filed: Feb. 26, 2019

Related U.S. Application Data
(63) Continuation-in-part of application No. PCT/CN2017/103367, filed on Sep. 26, 2017.

(51) Int. Cl. A61K 39/00; C07K 14/705; C07K 14/725; A61P 35/00

(52) U.S. Cl. CPC A61K 39/001111; C07K 14/70596; C07K 14/70517; A61K 2039/5156; C07K 14/70521; A61P 35/00; C07K 2319/03; C07K 14/7051

(57) ABSTRACT

A chimeric antigen receptor targeting to CD47, its encoding sequence, and its modified immune response cells, and the preparation and application thereof. The present invention constructs a chimeric antigen receptor targeting to CD47 and its modified immune response cells based on the SIRPα molecule, and the novel modified immune response cells can effectively target a variety of tumor cells, and can be used to prepare preparations for the treatment of tumors, especially the preparations for inhibiting the expression of CD47-positive tumor cells. The preparation of the modified immune response cells of the target CD47 is easy, and the modified immune response cells of the target CD47 have high killing rate on tumor cells.

### CROSS REFERENCE TO RELATED APPLICATIONS

This application is the Continuation-in-part of International Application No. PCT/CN2017/103367, filed on Sep. 26, 2017, the entire contents of which are incorporated by reference.

### TECHNICAL FIELD

The invention belongs to the field of tumor immunotherapy biological medicine technology and involves specific chimeric antigen receptor (CAR) T cells. In particular, it relates to a specific targeting to CD47 comprising SIRPα protein or its variants. Chimeric antigen receptor (CAR) and its modified immune response cells, as well as their preparation methods and applications.

### BACKGROUND

With the rapid development of biotechnology, immune cell therapy has become the fourth major therapy in the field of cancer therapy.

Cancer immunotherapy comprises adoptive cell therapy, immunomodulator, tumor vaccine and immunoassay, immunology checkpoint block therapy and so on. Among them, in the field of cell therapy, chimeric antigen receptor modified immune cells (especially Chimeric Antigen Receptor T-Cell (CAR-T) therapy has undoubtedly become a superstar for research institution and pharmaceutical industry. The company is competing for the star.

CAR-T (chimeric antigen receptor modified T cells) was used as a substitute. The principle of immune therapy is to modify T cells from patients themselves by means of genetic engineering. The antigen receptor is modified to generate CAR-T cells, which can specifically recognize tumor surface associated antigens. The engineering T cells can specifically recognize tumor surface antigen (tumor cell marker), thus targeting to the tumor. Relative to conventional immune cells, CAR-T cells that it has higher targeting, killing activity and persistence, and can overcome the local immunosuppressive microenvironment of tumor. And the state of host immune tolerance is broken. The modified immune cell therapy represented by CAR-T cells is urgent. The treatment of leukemia and non Hodgkin's lymphoma has a significant progresses and is regarded as the most promising therapy approach. One of the methods of tumor treatment.

However, 90% of cancers are solid tumor, more solid tumors and more tumor specific targets. Tumor specific antigens need to be further confirmed. The remarkable bottleneck of CAR-T immunotherapy for solid tumors is CAR-T cells require very high specificity of antigen expression on tumor cells, otherwise, T cells can be easily held. Continuous activation can kill normal cells or release a large number of cytokines, causing serious cytokine reaction effects. Although CAR-T exempts the specificity of pestilence therapy for tumor cell antigen expression is very high, but tumor specific target antigens are rare. Most of the antigens expressed by tumor does not have tumor only specificity, and tumor related resistance is not. CAR-T immunotherapy as a target has problems such as “miss the target”. Study the broad-spectrum, efficient and safe CAR-T immunotherapy is an urgent need.

The key to the application of chimeric antigen receptor modified immune response cells is to identify at least one tumor associated antigens, which are highly expressed on the surface of tumor cells but not expressed or very low on the surface of normal cells.

The human body has two different immune systems, one is the acquired immune system represented by T cells and another is the congenital immune system represented by macrophages. Macrophages play important roles in cell phagocytosis, antigen presentation, immune response, cell homeostasis, pathogen defense and antitumor immune regulation. However, the relationship between tumor-associated macrophages and tumors seems to be more complex: macrophages outside the tumor tissue can kill tumor cells directly and the more macrophages inside the tumor tissue has the worse killing effect on the tumor. Further studies showed that macrophages in tumor tissue can secrete growth factors to nourish tumor cells, promote tumor angiogenesis, and lead to tumor invasion and metastasis (McCracken et al (2017) Clin Cancer Res 21:3597-3601; Weiskopf Kipp (2017) European Journal of Cancer 76:100-109). CD47 expression was up-regulated in circulating hematopoietic stem cells and leukemic cells. The binding of CD47 on the cell surface with SIRPα on macrophages inhibited normal phagocytosis and escaped phagocytosis by macrophages.

Macrophage-mediated programmed cell clearance is based on the “eat me” signal and is an important mechanism for clearing disease and damaged cells before programmed cell death. Relative to it is “do not eat me” signal, such as CD47/SIRPα signal. The study also showed how macrophages recognize and phagocytize tumor cells by “eat me” and “don't eat me” signals: tumors mostly express calreticulin (CRT), a member of the “eat me” signal that recruits macrophages to phagocytize and kill tumor cells. However, in the course of tumor evolution, some tumor cells also express a molecule CD47, which sends out the signal of “don't eat me” and counteracts the signal of calcium reticulin “eat me”. Thus, when the expression of CD47, the representative of “don't eat me” signal, is up-regulated in tumor cells, macrophages in tumor tissues will be “blindfolded” to play an immune escape role. CD47 is an immunoglobulin-like protein widely expressed on the surface of cell membrane. It is also called integrin-associated protein (IAP) because of its function and integrin-related. CD47 was first found as a tumor antigen in human ovaries in the 1980s. Since then, CD47 has been found to be highly expressed in a variety of diseases including acute myeloid leukemia, chronic myeloid leukemia, acute lymphocytic leukemia, non-Hodgkin's lymphoma, multiple myeloma, bladder cancer and other solid tumors. In 1999, scientists discovered that CD47 binds to the ligand signal regulator alpha (SIRPα), which is expressed on the surface of macrophages, and sends out “don't eat me” inhibitory resistance signals, which enable tumor cells to escape the phagocytosis of macrophages, inhibit the innate immune system, induce cell fusion and down-regulate T cells. The expression of IL-12 receptor decreases the responsiveness of T cells to IL-12 and affects the activation of T cells.

In recent years, more and more studies have shown that blocking the binding of CD47 to SIRPα by antibodies in vitro can promote the phagocytosis of macrophages to tumor cells and induce apoptosis of tumor cells. In vivo, blocking CD47 with antibodies or a SIRPαFc fully human recombinant fusion protein has been shown to inhibit tumor growth in several mouse models (Majeti et al (2009) Cell 138: 286-299; Petrova et al (2017) Clin Cancer Res 23:1068-1079). Although the underlying molecular mechanisms are still not fully understood, CD47 has gradually become a new target for cancer treatment, and can provide a new means to conquer malignant tumors in the treatment process. Therefore, using the high expression of CD47 target on the surface of many tumor cells and its binding ability with SIRPα, we have constructed a new type based human SIRPα CAR-T cells with highly killing efficiency for tumor therapy.

### Technical Problems

In view of the above problems and/or other problems related to the technology, the aim of the invention is to overcome the existing tumors. The specificity of T cells binding and killing tumor cells is not strong in clinical environment. The problem of low efficiency is to provide chimeric antigen receptors targeting the human CD47, and their genes and recombinant expression vectors. Targeting to human CD47 protein engineered CAR-T cells and their applications. Targeting to human CD47 protein engineered CAR-T cells have the highly killing efficiency to tumor cells. This provides a promising way for cancer therapy.

### A Solution to the Problem

### SUMMARY

In the first aspect, this application provides the human SIRPα protein or its functional variants targeting the human CD47, wherein functional variants comprise sequences selected from the following groups:

(1) SEQ ID NO:4, SEQ ID NO:5, SEQ ID NO:6, SEQ ID NO:7, SEQ ID NO:8, or the amino acid sequence shown in SEQ ID No.9, or (2) (1) functional variants generated by modifications at one or more amino acids, wherein the amino acid sequences of the variants have 70%˜99% identity with the amino acid sequences shown by the SEQ ID NO:4, or SEQ ID NO:5, or SEQ ID NO:6, or SEQ ID NO:7, or SEQ ID NO:8, or SEQ ID NO:9.

In second aspect, this application provides a specific chimeric antigen receptor targeting the human CD47, wherein it comprises a guided sequence, an extracellular domain targeting the human CD47, a transmembrane domain, and an intracellular signaling domain connected are sequentially connected from the amino terminal to the carboxyl terminal, wherein the immune response cells modified by the SIRPα protein targeting the human CD47 have the killing efficiency up to 50%-90% when effector to target ratio is equal to 5:1, and the extracellular recognition domain of targeting the human CD47 comprises human SIRPα protein targeting the human CD47 or its functional variants, and preferably the human SIRPα protein targeting the human CD47 is the CD47 receptor.

In some embodiments, the specific chimeric antigen receptor also comprises a hinges region.

In some embodiments, the transmembrane domain comprises transmembrane regions.

In some embodiments, the intracellular signaling domain comprises the receptor tyrosine activating motif and costimulatory signaling domain. In some embodiments, the amino acid modifications include, but are not limited to, substitutions, deletions, and additions of amino acids including, but not limited to, derived polypeptides produced by substitutions, deletions, and additions of amino acids.

In some embodiments, the transmembrane domain comprises CD8 transmembrane, CD28 transmembrane, CD3 zeta transmembrane, CD4 transmembrane, 4-1BB transmembrane, OX40 transmembrane, ICOS transmembrane, CTLA-4 transmembrane, PD-1 transmembrane, LAG-3 transmembrane, 2B4 transmembrane, BTLA transmembrane, and any kind of synthetic peptides (not based on proteins associated with immune responses).

In some embodiments, the immune receptor tyrosine activating motif comprises the intracellular signaling domain of the CD3 zeta or the FcεRIγ intracellular signaling domain.

In some embodiments, the costimulatory signaling domain comprises one of kind CD28 intracellular signaling domain, CD137/4-1.BB intracellular signaling domain, CD134/OX40 intracellular signaling domain and ICOS intracellular signaling domain. In one embodiment, the hinge region or hinge region of the immunoglobulin is modified.

In one embodiment, the leader sequence is selected from SEQ ID NO:3.

In some embodiments, the amino acid sequence of human CD8 in the hinge region is selected from SEQ ID NO:10.

In some embodiments, the amino acid sequence of human CD8 in the transmembrane domain is selected from SEQ ID NO:11.

In some embodiments, the human 4-1BB intracellular domain is selected from SEQ ID NO:12.

In some embodiments, the human CD28 zeta intracellular domain is selected from SEQ ID NO:13.

In some embodiments, the CD3 zeta intracellular domain is selected from SEQ ID NO:14.

In some embodiments, the CD3 zeta intracellular domain is selected from SEQ ID NO:15.

In some embodiments, the chimeric antigen receptor targets the human CD47 which is expressed by recombination or vector.

In some embodiments, the amino acid sequence from the amino terminal to the carboxyl terminal of the chimeric antigen receptor comprises a guiding sequence, a human SIRPα sequence according to claim 1, a human CD8 hinge region sequence, a human CD8 transmembrane region sequence, a human CD28 intracellular domain sequence, a human 4-1BB intracellular domain sequence, and a CD3 zeta intracellular domain sequence which are sequentially connected.

In some embodiments, the amino acid sequence from the amino terminal to the carboxyl terminal of the chimeric antigen receptor comprises a guided sequence, a human SIRPα sequence according to claim 1, a CD8 hinge region sequence, a CD8 transmembrane region sequence, a 4-1BB intracellular domain sequence, and a CD3 zeta intracellular domain sequence which are sequentially connected.

In some embodiments, the amino acid sequence from the amino terminal to the carboxyl terminal of the chimeric antigen receptor comprises a guided sequence, and a human SIRPα sequence according to claim 1, a CD8 hinge region sequence, a CD8 transmembrane region sequence, CD28 intracellular domain sequence, and a CD3 zeta intracellular domain sequence which are sequentially connected.

In some embodiments, the amino acid sequence from the amino terminal to the carboxyl terminal of the chimeric antigen receptor comprises a guided sequence, a human SIRPα sequence according to claim 1, a CD8 transmembrane region, a 4-1BB intracellular domain sequence, and a CD3 zeta intracellular domain sequence which are sequentially connected.

In some embodiments, the amino acid sequence from the amino terminal to the carboxyl terminal of the chimeric antigen receptor comprises a guided sequence, a the human SIRPα sequence according to claim 1, a CD8 transmembrane region sequence, a CD28 intracellular domain sequence, and a CD3 zeta intracellular domain sequence which are sequentially connected.

In a preferred embodiment, the guide sequence is shown by SEQ ID NO:3.

In some preferred embodiments, the human SIRPα sequence is shown by any one of SEQ ID NO:4, SEQ ID NO:5, SEQ ID NO:6, SEQ ID NO:7, SEQ ID NO:8 or SEQ ID NO:9.

In a preferred embodiment, the amino acid sequence of human CD8 hinge region is shown by SEQ ID NO:10.

In a preferred embodiment the amino acid sequence of human CD8 transmembrane region is shown by SEQ ID NO:11.

In a preferred embodiment, the amino acid sequence of human 4-1BB intracellular domain is shown by SEQ ID NO:12.

In a preferred embodiment, the amino acid sequence of CD28 intracellular domain d is shown by SEQ ID NO:13.

In a preferred embodiment, the amino acid sequence of CD3 zeta intracellular domain is shown by SEQ ID NO:14 or SEQ ID NO:15.

In the third aspect, this application provides a plurality of nucleic acid molecules of encoding the specific chimeric antigen receptors targeting the human CD47 in the second aspect, wherein the nucleic acid molecules comprise a nucleotide sequence of the guiding sequence, a nucleotide sequence of human SIRPα protein targeting the human CD47, and the a guiding sequence of transmembrane domain, and a nucleotide sequence of intracellular signaling domain, wherein the transmembrane domain comprises a transmembrane region, the intracellular signaling domain comprises an immune receptor tyrosine activation sequence and a costimulatory signaling domain connected sequentially from 5 ‘to 3’, and optionally, the nucleic acid molecule also comprises the nucleotide sequence encoding hinges region.

In some embodiments, the intracellular signaling domain comprises the receptor tyrosine activating motif and costimulatory signaling domain.

In some embodiments, chimeric antigen receptor targeting the human CD47, wherein the nucleic acid molecule comprises the encoding sequence of hinge region.

In some embodiments, the transmembrane domain comprises transmembrane regions.

In some embodiments, the nucleic acid molecules comprise a nucleotide sequence encoding a guiding sequence, a nucleotide sequence encoding human SIRPα targeting the human CD47 according to claim 1, a nucleotide sequence encoding human CD8 hinge region, a nucleotide sequence encoding human CD8 transmembrane region, a nucleotide sequence encoding CD28 intracellular domain, a nucleotide sequence encoding human 4-1BB intracellular domain, and a nucleotide sequence encoding CD3 zeta intracellular domain sequentially connected from 5 ‘to 3’.

In some embodiments, the nucleic acid molecules comprise a nucleotide sequence encoding a guiding sequence, a nucleotide sequence encoding human SIRPα targeting the human CD47 according to claim 1, a nucleotide sequence encoding human CD8 transmembrane region, a nucleotide sequence encoding human 4-1BB intracellular domain, and a nucleotide sequence encoding CD3 zeta intracellular domain sequentially connected from 5 ‘to 3’.

In some embodiments, the nucleic acid molecules comprise a nucleotide sequence encoding a guiding sequence, a sequence encoding human SIRPα targeting the human CD47 according to claim 1, a nucleotide sequence encoding human CD8 hinge region, a nucleotide sequence encoding human CD8 transmembrane region, a nucleotide sequence encoding human 4-1BB intracellular domain, and a nucleotide sequence encoding CD3 zeta intracellular domain sequentially connected from 5 ‘to 3’.

In some embodiments, the nucleic acid molecules comprise a nucleotide sequence encoding a guiding sequence, a sequence encoding human SIRPα targeting the human CD47 according to claim 1, a nucleotide sequence encoding human CD8 transmembrane region, a nucleotide sequence encoding CD28 intracellular domain, and a nucleotide sequence encoding CD3 zeta intracellular domain sequentially connected from 5 ‘to 3’.

In some embodiments, the nucleic acid molecules comprise a nucleotide sequence of encoding a guiding sequence, a nucleotide sequence encoding human SIRPα targeting the human CD47 according to claim 1, a nucleotide sequence encoding human CD8 hinge region, a nucleotide sequence encoding human CD8 transmembrane region, a nucleotide sequence encoding CD28 intracellular domain, and a nucleotide sequence encoding CD3 zeta intracellular domain sequentially connected from 5 ‘to 3’.

In the fourth aspect, this application provides a recombinant vector or expression plasmid comprising the nucleic acid of the third aspect of this application.

In the fifth aspects, this application provides a promoter for constructing the recombinant vector in the fourth aspect, and expressing the specific chimeric antigen receptor targeting the human CD47 described in the second aspect of this aspect. The promoters include but are not limited to, nucleotide sequences, such as EF1 Alpha promoter showed as SEQ ID NO:29 and the EFS promoter showed as SEQ ID NO:30.

In a preferred embodiment, the promoter is a nucleotide sequence, such as the EFS promoter shown in SEQ ID NO:30.

In a preferred embodiment, the nucleotide sequences is showed as EF1 alpha promoter of SEQ ID NO:29.

In the sixth aspect, the application provides the recombinant virus which can express the chimeric antigen receptors specifically targeting to CD47 in the second aspect of this application are able to infect immune response cells.

In the seventh aspect, this application provides a separate modified immune response cell, which comprises a chimeric antigen receptor targeting the human CD47 in the second aspect of this application and the recombinant vector or expression vector from transformation of is described in the fourth aspect of the invention.

In some embodiments, the immune response cells may comprise the cytotoxic T lymphocytes, NK cells, and NKT cells or helper T cells.

In some embodiments, the immune response cells also comprise at least one exogenous costimulatory ligand.

In the eighth aspect, this application provides a preparation method for the CAR-T cells in the seventh aspect of the application, wherein the method comprises the following steps. First, ligated the nucleic acid molecules according into claim 5 into the initial expression vector by molecular cloning, and acquired the expression vector for expressing the specific chimeric antigen receptor targeting to CD47. Then, transduced the expression vector of specific chimeric antigen receptor targeting to CD47 into 293T cells to obtain virus solution, and generated the immune response cells by expressing specific chimeric antigen receptor targeting to CD47 were produced from the infected cells with virus solution.

In some embodiments, the immune response cells are selected from the T cells, natural killer (NK) cells, Toxic T lymphocytes (CTL), regulatory T cells, human embryonic stem cells and pluripotent stem cell which could be differentiated to lymphoid cells, T cells or natural killer (NK) cells are preferred.

In the ninth aspect, the application provides a pharmaceutical composition used for the treatment or prevention of a tumor diseases, malaises, or health disorder, which comprises the effective dose and pharmaceutically acceptable dose of CAR-T cells, wherein the diseases, malaises, or health disorder are related to the specific interaction between human SIRPα protein and its ligand CD47, wherein the disease, malaises or health disorder of comprise pathogen infection, autoimmune disease, inflammatory disease, allograft, graft rejection and senescence.

In the tenth aspect, the application provides a kit used for the treatment or prevention of a tumor diseases, malaises, or health disorder, which comprises the effective dose and pharmaceutically acceptable dose of CAR-T cells, wherein the diseases, malaises, or health disorder are related to the specific interaction between human SIRPα protein and its ligand CD47, and wherein the disease, malaises or health disorder of comprise pathogen infection, autoimmune disease, inflammatory disease, allograft, graft rejection and senescence, and wherein the treatments and the preventions involve the infusion of the effective quantity chimeric antigen receptor T cells (CAR-T cells) targeting into patients with diseases that are caused by specific interactions between human SIRPα protein and its ligand CD47 in the seventh aspect of the application, and wherein malaises or disorders of health are related to the specific interaction between human SIRPα protein and its ligand CD47.

In some embodiments, diseases, malaises or health disorders comprise tumor formation, infection, autoimmunity, allograft, graft rejection, and aging.

The invention is based on human SIRPα molecule to construct specific chimeric antigen receptor targeting the human CD47 or the CAR modified T cell (CAR-T cell), the innovative CAR-T cells can effectively target to attack many kinds of tumor cells which can be developed preparations for the treatment of tumors, especially the expression of CD47 positive tumor cells.

### Beneficial Effect

Inventor of the invention accidentally discovered in the research that the invention comprises the specific construction of human SIRPα chimeric antigen receptor targeting the human CD47 modified T cells, the preparation method is simple. At the effector to target ratio is 10:1, the killing rate of tumor cells from 40% to 70%, and also can significantly prolong persistence of the immune cells in patients. The improvement of immune cells specifically target tumor cells, especially those CD47 positive. The improvement of immune cells or their composition cytotoxicity for tumor or tumor formation from liver cancer, gliblastoma, ovarian cancer, gastric cancer, medulloblastoma, pancreatic cancer, lung cancer, prostate cancer, breast cancer, neuroblastoma, bladder cancer, colon cancer, renal cell carcinoma, leukemia, lymphoma, multiple myeloma, melanoma.

The modified immune response cells that express human chimeric SIRPα receptor targeting to CD47 positive tumors are described in the invention which is new approach option for tumor immunotherapy, and has potential industrial application prospects.

### BRIEF DESCRIPTION OF THE DRAWINGS

FIG. 1 is a schematics of lentivirus vector used in the present invention as an example.

FIGS. 2A and 2B are a connection sequential schematics of the construction of the chimeric antigen receptors in embodiments 1 and 2. FIG. 2A. EF1 alpha promoter (sequence showed in table as SEQ ID NO:29), FIG. 2B. EFS promoter (sequence showed in table as SEQ ID NO:30), wherein the amino acid sequence of human CD3 zeta domain is described as marker 1, the amino acid sequence of human 4-1BB intracellular domain is described as marker 2, and the amino acid sequence, wherein the amino acid sequence of human CD8 transmembrane region is described as marker 3, and CD8 hinge region amino acid sequence is described as marker 4, human SIRPα amino group, acid sequence is described as marker 5, the guidance amino acid sequence is described is described as marker 6.7 shows EF1 alpha promoter whereas 8 is EFS promoter.

FIG. 3 depicts the result of T cell purity analyzed by flow cytometry in embodiment 2.

FIGS. 4A, 4B and 4C depict the result of CAR-T cell activity detected by flow cytometry in embodiment 5, wherein FIG. 4A is a blank control which means T cell without infection with virus, FIG. 4B is specific CAR-T targeting to CD19 KD-019 control, FIG. 4C is a specific CAR-T cell targeting to CD47 KD-045.

FIGS. 5A, 5B and 5C depict the expression of KD-045 CAR molecules detected by flow cytometry in embodiment 5, wherein FIG. 5A is a blank control which means T cell without infection with virus, FIG. 5B is specific CAR-T targeting to CD19 KD-019 control, FIG. 5C is a specific CAR-T cell targeting to CD47 KD-045.

FIG. 6 depicts a specific CAR-T targeting to CD47 positive glioblastoma U251 cells, which shows the results of the killing activity to the target tumor cells in this application.

FIG. 7 depicts a specific CAR-T targeting to CD47 positive medulloblastoma HTB186 cells, which shows the results of the killing activity to the target tumor cells in this application.

FIG. 8 depicts a specific CAR-T targeting to CD47 positive acute myeloid leukemia U937 cells, which shows the results of the killing activity to the target tumor cells in this application.

FIG. 9 depicts a specific CAR-T targeting to CD47 positive hepatoma SMMC7721 cells, which shows the results of the killing activity to the target tumor cells in this application.

FIG. 10 depicts a specific CAR-T targeting to CD47 positive pancreatic cancer BXPC3 cells, which shows the results of the killing activity to the target tumor cells in this application.

FIG. 11 depicts a specific CAR-T targeting to CD47 positive colorectal cancer HCT116 cells, which shows the results of the killing activity to the target tumor cells in this application.

FIGS. 12A and 12B depict the killing results of the tumor xenograft animal model by subcutaneous infusing a specific CAR-T targeting to CD47 positive glioblastoma U251 cells, wherein the growth of subcutaneous transplantation tumor in xenograft NSG mice is detected as FIG. 12A shown whereas the fluorescence intensity of the tumor is detected as FIG. 12B shown.

FIGS. 13A and 13B depict the killing results of the tumor xenograft animal model by subcutaneous infusing a specific CAR-T targeting to CD47 positive medulloblastoma HTB186 cells, wherein the growth of subcutaneous transplantation tumor in xenograft NSG mice is detected as FIG. 13A shown whereas the fluorescence intensity of the tumor is detected as FIG. 13B shown.

FIGS. 14A and 14B depict the killing results of the tumor xenograft animal model by subcutaneous infusing a specific CAR-T targeting to CD47 positive acute myeloid leukemia U937 cells, wherein the growth of subcutaneous transplantation tumor in xenograft NSG mice is detected as FIG. 14A shown whereas the fluorescence intensity of the tumor is detected as FIG. 14B shown.

FIGS. 15A and 15B depict the safety tolerance dose and toxicology test results by using B-NSG animals with KD-045 CAR, wherein FIG. 15A shows toxicological test results, and FIG. 15B shows the test results of a safe tolerance dose experiment.

FIGS. 16A, 16B, 16C and 16D depict the cytokine IFN-γ release results of KD-045 CART targeting to tumor cell lines U251, HTB186, U937 and SMMC7721 in vitro, respectively.

### DETAILED DESCRIPTION OF THE EMBODIMENTS

Unless otherwise defined, all technical and scientific terms used in this article are technical personnel in the field of the invention. The meaning that is usually understood.

Term “functional variant” is the term “functional variety” that is modified by the maternal structure. With the same or similar biological functions and properties, such as those having the same target binding function with the maternal body. Variant. As a particular example, functional variants can be carried out in one or more places in the matrix. The replacement is obtained. The functional variant in this application is the amino acid sequence of the ligand SIRPα in CD47. Based on the modified structure produced by targeting CD47 targets.

Term “amino acid modification” refers to CAR which does not significantly affect or alter the disclosure of the amino acid sequence, i.e., the extracellular recognition domain is characterized by a conservative amino acid modification. This conservative modification comprises the amino acid substitution, addition and deletion.

Term “homology” refers to the amino group of the target amino sequence or the target nucleotide sequence matches with the reference sequence with a high proportions homology of acid or nucleotides. The homology in this application can be produced using standard software such as BLAST or FAST.

Term of chimeric antigen receptor (CAR).

The chimeric antigen receptor comprises the guiding peptide, extracellular target recognition domain, transmembrane domain and intracellular domain.

Specific chimeric antigen receptor (CAR) is a specific antibody or ligand designed artificially to bind target antigens, i.e., a specific chimeric antigen receptor (CAR) targeting the human CD47 in this application. CAR could integrate specifically coding sequences into T cells through lentiviral vectors, i.e., transferring the coding sequence of monoclonal antibodies into T cells.

Term “antigen” refers to the highly specific expression of a target molecule in the surface of tumor cells, which can be identified and targeted specifically with ligand. In this invention, it refers to the human CD47. The extracellular antigen binding domain is CD47 in specific exemplary embodiments. Any SIRPα molecule sequence is possible to exist in heterogeneous sequences of fusion protein to form extracellular antigen binding domain in specific particular embodiments.

The receptor of CD47 protein is SIRPα and SIRPα-CD47 system plays an important role in anti-tumor immunity of the body. SIRPα targets CD47 produced on the surface of tumor cells and transduce the activation function of immune system, and thereby killing the tumor cells.

Term “recognition” refers to the selective combination of targets. The term “specific combination” or “specific combining” “or” specific targeting” is used in this article which means that peptides or their fragments recognize and bind to the target biomolecules, such as peptides, but it basically does not recognize the binding of other molecules in the sample, i.e., naturally including other molecules of biological sample of the polypeptide of the invention.

Term “specific binding” refers to the combination of two molecules, such as ligand and receptor, which are very characteristic. When there are many other molecules, one molecule (ligand) and another specific molecule (receptor). The ability to combine, that is, to show the preferential combination ability of one molecule to another molecule in the heterogeneous mixture of molecules. The specific binding of ligand to receptor is also verified as follows: when there is an excess of unlabeled ligands, the binding of the labelled ligand to the receptor decreased (i.e., the competition test).

The term “receptor” is a molecular structure that can be specifically combined with target sequences. In particular, the receptor is combined with another the ligand on the cell surface allows cell to cell recognition and/or interaction. The receptor is the SIRPα targeting the human CD47 in the present invention.

Term “costimulatory molecule” refers to a molecule on the cell surface except antigen receptors or their ligands which affects lymphocytes recognizing antigens.

Costimulatory ligand is a protein expressed on the cell surface, which produces a co stimulation response when it combines with its receptor, that is, the intracellular response to the stimulation of the antigen when the antigen is combined with its CAR molecule.

Term “vector” refers to any genetic element, such as plasmid, phage, transposon, clay, chromosome, viruses, virus particles, etc., which can be duplicated under appropriate control elements, and transfers gene sequence to the target cell. Therefore, the term comprises cloning and expression vectors, as well as viral vectors and plasmid.

Term “expression vector” refers to a recombinant nucleic acid sequence, that is, recombinant DNA molecule, which comprises the coding sequence and nucleic acids sequence necessary for expressing the coding sequences in specific host organisms. The nucleic acid sequences necessary for expression in prokaryotes typically comprise promoters, operon (optional) and ribosomal binding sites are usually accompanied by other sequences whereas eukaryotic cells utilize promoters, enhancers, terminator and polyadenosine acidification signal.

The term “immune response cell” used in this article is the cell or its progenitor or its progeny cells that play important role in the immune response.

The term “isolation cells” refers to the immune cells that are isolated from the molecules and/or cell components of natural cells mixture.

The term “pathogen” used in this article refers to Viruses, bacteria, fungi, parasites, or protozoans that can cause diseases.

The term “(treating, treatment)” refers to clinical intervention that attempts to change the process of disease of an individual or cell that is being treated, and can be used for prevention or during the process of clinical pathology. The therapeutic effect of treatment comprise, but not limited to, the occurrence or recurrence of the disease, the remission of the symptoms, the reduction of any direct or indirect pathological consequences of the disease, the prevention of metastasis, the speed of reducing the progression of the disease, the reduction or mitigation of the state of the disease, and the prognosis of remission or improvement. By preventing the progress of disease or disease, treatment can prevent the deterioration caused by the patients who have been attacked or diagnosed or suspected to suffer from the disease, and the treatment can prevent the disease or symptoms of the subjects who have the risk of the disease or those who are suspected to have the disease.

The term “killing efficiency” refers to a certain ratio of tumor cell dead after tumor antagonists and tumor cells are mixed in the in vitro testing system. In this article, the ratio of tumor cell death caused by chimeric antigen receptor modified immune response cells. In particular, the killing ratio of the tumor cells expressing CD47 is described when SIRPα chimeric antigen receptor modified T cells at the ratio of effector to target 5:1.

The term “disease” used in this article is any disease or disorder that destroys or interferes with the normal functions of cells, tissues, or organs such as diseases comprise tumor formation or cell pathogen infection.

The term “effective dose” used in this article refers to the amount enough to have therapeutic effect. In one embodiment, effective dose is the amount enough to prevent, improve, or suppress the growth, continuous proliferation, or metastasis of a tumor formation, i.e., invasion or migration.

The term “exogenous nucleic acid molecules or peptides” used in this article, refers to nucleic acid molecules or peptides that are not normally found in or obtained by cells. (i.e., cDNA, DNA or RNA molecules). The nucleic acid may be derived from another organism, or it can be an mRNA molecule that is not normally exist in cells or samples.

In the first aspect, this application provides a human SIRPα protein targeting the human CD47 and its functional variants, which include the following:

(1) SEQ ID NO:4, SEQ ID NO:5, SEQ ID NO:6, SEQ ID NO:7, SEQ ID NO:8, or SEQ ID NO:9 the amino acid sequence shown as (1), or (2) the amino acid modified variants of (1), wherein the amino acid sequences of the variants have 70%-99% identity to the amino acid sequences shown by the SEQ ID NO:4, or SEQ ID NO:5, or SEQ ID NO:6, or SEQ ID NO:7, or SEQ ID NO:8, or SEQ ID NO:9.

According to the existing researches, the peptide SIRPα protein targeting the human CD47 is based on the amino acid sequence of human SIRPα protein.

According to the existing researches, herein CAR constructed based on the full length amino acid sequence of SIRPα only have poor function, because the functional domains such as the SIRPα intracellular region is derived from natural killer cells and is not compatible with the T cells.

Then, the inventor screened and analyzed SIRPα amino acid sequence targeting to CD47 based on the full length amino acid sequence of SIRPα by using protein prediction tool for sequence analysis and the functional domain optimization, and further designed and constructed the amino acid sequence of chimeric antigen receptor, which comprises the specific part sequence of SIRPα targeting the human CD47, wherein it is also a technical challenge for the skilled person in this field to design and develop the amino acid sequences of the hinge area, transmembrane region, and intracellular signaling activation region most suitable with and matched with T cells.

Through the creative work and continuous amino acid sequence design and sequence assembly and screening, the inventor conducted random screening test and specific function verification, such as constructing viral vectors and further infection of T cells to obtain modified T cells, and detecting the cytotoxic activity of modified T cell in vitro, the sequences of more than 10 different CAR molecules are screened by, the sequences were then adjusted according to the comparison and blast of the results of multiple random composition, and finally the best sequence was screened out. The SIRPα amino acid sequence and its functional variants at the high titer targeting the human CD47 of the invention were obtained.

In some particular embodiments, the amino acid modifications include, but are not limited to substitutions, deletions, and additions of amino acids, the functional variants include, but are not limited to the derived peptides or peptide analogues of the human SIRPα amino acid sequences targeting the human CD47 produced by the substitutions, deletions, and additions of amino acids, and the derived peptides and peptide analogues are homologous to the amino acid sequences of human SIRPα.

In some particular embodiments, the amino acid modifications include, but are not limited to, chemical modifications of the amino acid side chain, natural or unnatural amino acid substitutions, mutations, deletions, insertions, or the post-translational modifications.

In some particular embodiments, the variants of amino acid produced by modifications at one or more points in the amino acid sequences of the SIRPα amino acid sequence targeting the human CD47 are the polypeptides homologous to the amino acid sequence shown by SEQ ID NO:5. In some particular embodiments, the variant of amino acid modified by one or more amino acid sequences of the SIRPα amino acid sequence targeting the human CD47 is a polypeptide that is homologous to the amino acid sequence shown by SEQ ID NO:6. In some particular embodiments, the variants of amino acid produced by modifications at one or more points in the amino acid sequences of the SIRPα amino acid sequence targeting the human CD47 are the polypeptides homologous to the amino acid sequence shown by SEQ ID NO:7. In some particular embodiments, the variants of amino acid produced by modifications at one or more points in the amino acid sequences of the SIRPα amino acid sequence targeting the human CD47 are the polypeptides homologous to the amino acid sequence shown by SEQ ID NO:8. In some particular embodiments, the variants of amino acid produced by modifications at one or more amino acid sequences of the SIRPα amino acid sequence targeting the human CD47 are the polypeptides homologous to the amino acid sequence shown by SEQ ID NO:9.

The homologous polypeptides in the first aspect determined compared with the reference polypeptide, herein these peptides refer to the polypeptides with 70%˜99% preferably 80%˜99%, more preferable 90%˜99%, and most preferably 95%˜99% identity to the SIRPα targeting the human CD47.

In some particular embodiments, the polypeptides with 70%˜99% preferably 80%˜99%, more preferable 90%˜99%, and most preferably 95%˜99% identity to the amino acid sequences set forth by SEQ ID NO:4, SEQ ID NO:5, SEQ ID NO:6 as described. SEQ ID NO:7, SEQ ID NO:8 or SEQ ID NO:9 are the polypeptides derived by substitution, deletion or addition of 1˜10 amino acids, preferably 1˜5 amino acids, more preferably 1˜3 amino acids from any one amino acid sequence among SEQ ID NO:4, SEQ ID NO:5, SEQ ID NO:6, SEQ ID NO:7, SEQ ID NO:8 or SEQ ID NO:9.

In addition to full-length polypeptides, the subject of the first aspect of the application also provides any fragment of a peptide or peptide domain in the topics for the purpose of this application. In some embodiments, the fragments may be at least 5˜15 amino acids. In some embodiments, the fragments may be at least 20 continuous amino acids, at least 30 continuous or at least 50 continuous amino acids. In some embodiments, the fragments may be at least 60 to 80, 100, 200, 300, or more continuous amino acids.

The amino acid fragments of the first aspect of the application may be produced by a method known by the skilled person in the field, or the non protein analogue may be produced by a processing strategy (e.g., removing the amino acids that is not needed for the biological activity from the new polypeptides or removing the amino acids by the alternative mRNA splicing or the alternative protein processing events).

In the second aspect, this application provides a specific chimeric antigen receptor targeting the human CD47 characterized in that, the chimeric antigen receptor comprises a guided sequence from the amino terminal to the carboxyl terminal, an extracellular domain targeting the human CD47, a transmembrane domain, and a intracellular signaling domain, wherein the immune response cells modified by the SIRPα protein targeting the human CD47 have the killing efficiency up to 50%˜90% when effector to target ratio is equal to 5:1, and the extracellular recognition domain targeting the human CD47 comprises human SIRPα protein or its functional variant targeting the human CD47 according to claim 1, and preferably the human SIRPα protein targeting the human CD47 is the CD47 receptor.

In some embodiments, the specific chimeric antigen receptor binding to human CD47 comprises hinge regions.

In some particular embodiments, a CAR further comprise a spacer region, which connects the antigen binding domain to the transmembrane domain. The spacers region may be sufficiently flexible to orients the antigen binding domains in different directions to facilitate antigen recognition. The spacer region may originate from the hinge region of IgG1, or the CH2CH3 region of immunoglobulin and the portion of CD3.

In some particular embodiments, CAR can comprise the spacer, which connects the antigen binding domain to the transmembrane domain. A spacer region can be flexible enough to allow antigen binding domains to orientate in different directions to facilitate antigen recognition. The spacer area can be from the hinge area of IgG1, or the CH2CH3 area of immunoglobulin and the part of CD3.

In some embodiments, the specific chimeric antigen receptor that targets to human CD47 comprises a hinge region.

In some embodiments, the intracellular signal domain comprises an immunoreceptor tyrosine activation motif, a costimulatory signal domain, an intracellular signal domain comprises an immunoreceptor activation motif and optionally one or more costimulatory signaling domains. The intracellular signaling domain comprises the immune receptor activation motif and optionally comprises one or more costimulatory signaling domains.

In some particular embodiments, the intracellular domain of CAR also comprises at least one costimulatory signaling transduction region, which comprises at least one costimulatory molecule that can provide optimal lymphocyte activation. The “costimulatory molecule” used in this article refers to the effect of lymphocytes on antigen, except for a cell surface molecule from antigen receptor or ligand. At least one costimulatory signaling transduction region comprise CD28 polypeptide, 4-1BB polypeptide and OX40 polypeptide, ICOS polypeptide, 2B4 polypeptide, BTLA polypeptide, synthetic peptide (not based on proteins associated with immune responses), or their composition. Costimulatory molecule can be combined with costimulatory ligands, and costimulatory ligands are proteins expressed on the cell surface, which produce a costimulatory response when they combine with their receptors, that is, the intracellular response to the stimulation of the antigen when the antigen is combined with its CAR molecule. Costimulatory ligands comprise, but are not limited to, CD80, CD86, CD70, OX40L, 4-1BBL, CD48, TNFRSF14 and PD-L1. As an embodiment, the 4-1BB ligand (4-1BBL) can combine 4-1BB (also known as “CD137”) to provide intracellular signals, which combine with CAR signals to induce the function of CAR+T cells.

In some embodiments, the costimulatory signaling transduction region of intracellular domain of CAR comprises two kinds of costimulatory molecules: CD28 and 4-1BB (see Sadelain et al., Cancer Discovery, OF1-11, (2013)), or CD28 and OX40.

In some particular embodiments, the intracellular domain of CAR comprises CD3 zeta polypeptides that activate or stimulate cells, such as lymphoid lineages, such as T cells. The CD3 zeta comprises three immune receptor tyrosine-based activation motif, and the activation signal is transmitted to the cell after binding antigen (for example, the lymphoid lineage cells, such as T cells).

In some particular embodiments the extracellular recognition domain (also referred to as the extracellular domain or simply referred by the recognition element contained therein) comprises a recognition elements that specifically binds to molecules and antigens present on the surface of the cell of the target cell. The function of the extracellular recognition domain is to anchor the binding cell membrane.

The extracellular recognition domain may also comprise a leader sequence or signal peptide that introduces a nascent protein into the endoplasmic reticulum. A signal peptide or leader sequence may be necessary if the CAR will be glycosylated and anchored in the cell membrane. The signal sequence or leader sequence may be a peptide sequence (about 5, about 10, about 15, about 20, about 25 or about 30 amino acids in length) present at the N-terminus of the newly synthesized protein, which directs the protein into the secretory pathway.

The domain of extracellular recognition antigen may also comprise the guide sequence or signal peptide that guides new proteins into the endoplasmic reticulum.

In some particular examples, the leader sequence is covalently linked to the 5-terminal of the extracellular antigen binding domain.

In some particular embodiments, the transmembrane domain comprises a transmembrane domain.

In some particular embodiments, the intracellular signaling domains comprises immune receptor tyrosine activating motif and costimulatory signaling domain. In some particular embodiments, the transmembrane domains of CAR comprise at least part of hydrophobic alpha helices. Different transmembrane domains produce different receptor stability. After antigen recognition, receptor clusters and signals are transduced to cells.

In some particular embodiments, the transmembrane domains of CAR may also comprise any kind of the CD8 transmembrane, the CD28 transmembrane, the CD3 zeta transmembrane, the CD4 transmembrane, the 4-1BB transmembrane, the OX40 transmembrane, the ICOS transmembrane, the CTLA-4 transmembrane, the PD-1 transmembrane, the LAG-3 trans membrane, the 2B4 transmembrane and the transmembrane region, any kind of synthetic peptides (not based on proteins associated with immune responses).

In some particular embodiments, the immune receptor tyrosine activating motif comprises intracellular signaling domain of the CD3 zeta or the FcεRI gamma intracellular signaling domain.

In some particular embodiments, the costimulatory signaling domain comprises at least one of the CD28 intracellular signaling domains, the CD137/4-1BB intracellular signaling domain, the CD134/OX40 intracellular signaling domain, and the ICOS intracellular signaling domain.

In some particular embodiments, the hinge is selected one from the hinges of the CD8 apha hinge, the IgG hinge, or the variants of the hinges or hinges of the whole or part of the immunoglobulin through modifications at one or more amino acids

In some particular embodiments, the intracellular domain of a CAR comprises a human CD3 zeta polypeptide that can activate or stimulate cells (eg, cells of the lymphoid lineage, such as T cells). CD3 zeta contains 3 ITAMs and transmits an activation signal to cells (eg, cells of the lymphoid lineage, such as T cells) upon binding to the antigen.

In some particular embodiments, the intracellular domain of CAR comprises CD3 zeta polypeptides that activate or stimulate cells, such as lymphoid lineages, such as T cells. The CD3 zeta comprises three Immune receptor tyrosine-based activation motif, and the activation signal is transmitted to the cell after binding antigen (for example, the lymphoid lineage cells, such as T cells).

In certain particular embodiments, the intracellular domain of the CAR further comprises at least one costimulatory signaling region which comprises at least one costimulatory molecule that provides optimal lymphocyte activation. The “costimulatory molecule” used in this article refers to the effect of lymphocytes on antigen, except for a cell surface molecule from antigen receptor or ligand. As used herein, “costimulatory molecule” refers to a cell surface molecule other than an antigen receptor or its ligand required for an effective response of a lymphocyte to an antigen. At least one costimulatory signaling region comprise a CD28 polypeptide, a 4-1BB polypeptide, an OX40 polypeptide, an ICOS polypeptide, a 2B4 polypeptide, a BTLA polypeptide, a synthetic peptide (not based on a protein associated with an immune response), or a combination thereof. A costimulatory molecule can bind to a costimulatory ligand, which is a protein expressed on the cell surface that, when bound to its receptor, produces a costimulatory response, ie, a cell that provides stimulation when the antigen binds to its CAR molecule. Should be inside costimulatory ligands include, but are not limited to, CD80, CD86, CD70, OX40L, 4-1BBL, CD48, TNFRSF14, and PD-L1.

In an exemplary embodiment, the costimulatory molecule is a 4-1BB ligand, (ie, 4-1BBL), which can bind to 4-1BB (also known as “CD137”) to provide an intracellular signal that is combined with the CAR signal to induce CAR Effector cell function of +T cells.

In some embodiments, the costimulatory signaling transduction region of intracellular domain of CAR comprises two kinds of costimulatory molecules: CD28 and 4-1BB (see Sadelain et al., Cancer discovery, OF1-11, (2013)), or CD28 and OX40. In some non-limiting embodiments, the hinge region are selected one from the group consisting of a CD8 alpha hinge region, an IgG hinge region, or the entire or portion of the variants of the immunoglobulin comprising one or more amino acid modifications in the hinge region or hinge region.

In one embodiment, the amino acid sequence of the chimeric antigen receptor from the amino terminus to the carboxy terminus comprises the leader sequence, the amino acid sequence of human SIRPα targeting the human CD47 described in the first aspect of the present application, and the human CD8 hinge region, the amino acid sequence, the amino acid sequence of the human CD8 transmembrane region, the amino acid sequence of the human CD28 intracellular domain, the amino acid sequence of the human 4-1BB intracellular domain, and the amino acid sequence of the human CD3 cell intracellular domain sequentially connected in series.

In a preferred embodiment, the amino acid sequence of the chimeric antigen receptor from the amino terminus to the carboxyl end comprises the leader sequence, the amino acid sequence of SIRPα targeting the human CD47 described in the first aspect of the present application, amino acid sequence of human CD8 transmembrane region, amino acid sequence of human 4-1BB intracellular domain and amino acid sequence of human CD3 zeta intracellular domain are sequentially connected in this application.

In a preferred embodiment, the amino acid sequence of the chimeric antigen receptor from the amino terminus to the carboxyl end comprises the leader sequence, the amino acid sequence of SIRPα targeting the human CD47 described in the first aspect of the present application, amino acid sequence of human CD8 transmembrane region, amino acid sequence of human CD28 intracellular domain and amino acid sequence of human CD3 zeta intracellular domain are sequentially connected in this application.

In one preferred embodiment, the amino acid sequence of the chimeric antigen receptor from the amino terminus to the carboxyl end comprises the leader sequence, the amino acid sequence of SIRPα targeting the human CD47 described in the first aspect of the present application, amino acid sequence of human CD8 hinge region, amino acid sequence of human CD8 transmembrane region, amino acid sequence of human 4-1BB intracellular domain and amino acid sequence of human CD3 zeta intracellular domain are sequentially connected in this application.

In one preferred embodiment, the amino acid sequence of the chimeric antigen receptor from the amino terminus to the carboxyl end comprises the leader sequence, the amino acid sequence of SIRPα targeting the human CD47 described in the first aspect of the present application, amino acid sequence of human CD8 hinge region amino acid sequence of human CD8 transmembrane region, amino acid sequence of human CD28 intracellular domain and amino acid sequence of human CD3 zeta intracellular domain are sequentially connected in this application.

In some particular embodiments, the guiding sequence is selected from a polypeptide with an amino acid sequence shown by SEQ ID NO:3.

In some particular embodiments, the CAR of the subject disclosed in the second aspect of this application comprises the spacer region, also known as the hinge region, which connects the antigen binding domain to the transmembrane domain. A spacer region can be flexible enough to allow antigen binding domains to orientate in different directions to facilitate antigen recognition. The spacer region may originate from the hinge area of IgG1, or the CH2CH3 area of immunoglobulin and the part of CD3. Some spacer regions comprise the immunoglobulin CH3 domain or the CH3 domain and the CH2 domain, while some spacer region comprise the whole or part of the immunoglobulin (for example, IgG1, IgG2, IgG3, IgG4) hinges, that is, the sequence locates between the CH1 and the CH2 domains of the immunoglobulin, such as the IgG4Fc hinges or CD8 hinges. The sequence of immunoglobulin sources can comprise one or more amino acid modifications, for example, 1, 2, 3, 4 or 5 substitutions. In some particular embodiments, the spacer area is hinge region.

In some particular embodiments, the subject covered by CAR in the second aspect of the transmembrane domain comprising CD28 polypeptide and the costimulatory signaling transduction region comprising CD28 polypeptide.

In some embodiments, hinge region comprises human CD8 polypeptide that is selected from a polypeptide with an amino acid sequence shown by SEQ ID NO:10.

In some embodiments, the transmembrane region comprises human CD8 peptides that is selected from a polypeptide with an amino acid sequence shown by SEQ ID NO:11.

In some embodiments, the amino acid sequence of the human 4-1BB intracellular domain is selected from a polypeptide with an amino acid sequence shown by SEQ ID NO:12.

In some particular embodiments, the intracellular domain of CAR can comprise CD3 zeta polypeptides that activate or stimulate cells, such as lymphoid lineages, such as T cells. The CD3 zeta comprises three Immune receptor tyrosine-based activation motif, and the activation signal is transmitted to the cell after binding antigen (for example, the lymphoid lineage cells, such as T cells).

In some embodiments, the amino acid sequence of the CD3 zeta intracellular domain is selected from a polypeptide with an amino acid sequence shown by SEQ ID NO:14.

In some particular embodiments, the amino acid sequence of the CD3 zeta intracellular domain is selected from is selected from a polypeptide with a sequence of amino acids shown by SEQ ID NO:15.

In some embodiments, the amino acid sequence of the human CD28 intracellular domain is selected from a polypeptide with an amino acid sequence shown by SEQ ID NO:13.

In an exemplary embodiment, the guiding sequence is shown as SEQ ID No.3.

In an exemplary embodiment, amino acid sequence of human SIRPα is described as SEQ ID NO:4. In an exemplary embodiment, amino acid sequence of human SIRPα is described as SEQ ID NO:5. In an exemplary embodiment, amino acid sequence of human SIRPα is described as SEQ ID NO:6. In an exemplary embodiment, amino acid sequence of human SIRPα is described as SEQ ID NO:7. In an exemplary embodiment, amino acid sequence of human SIRPα is described as SEQ ID NO:8. In an exemplary embodiment, amino acid sequence of human SIRPα is described as SEQ ID NO:9.

In an exemplary embodiment, amino acid sequence of human CD8 hinge region is described as SEQ ID NO:10.

In an exemplary embodiment, amino acid sequence of CD8 transmembrane region is described as SEQ ID NO:11.

In an exemplary embodiment, amino acid sequence of the intracellular domain of human 4-1BB is described as SEQ ID NO:12.

In an exemplary embodiment, amino acid sequence of CD28 domain is described as SEQ ID NO:13.

In an exemplary embodiment, amino acid sequence of the CD3 zeta domain is described as SEQ ID NO:14.

In an exemplary embodiment, amino acid sequence of the CD3 zeta domain is described as SEQ ID NO:15.

In one particular embodiments, specific chimeric antigen receptor (CAR) targeting the human CD47 is expressed in recombinant system or by vector.

In some particular embodiments, the intracellular domain of the CAR targeting the human CD47 comprises at least one costimulatory signaling conduction region, which comprises at least one of the costimulatory ligand molecules that activates lymphocyte. It is combined with CAR signal to induce the effector cell function of CAR-T cells, wherein the costimulatory ligands comprise but are not limited to CD80, CD86, CD70, OX40L, 4-1BBL, CD48, TNFRSF14 and PD-L1.

In one particular embodiments, the intracellular domain of CAR targeting the human CD47 in this application comprises 4-1BB polypeptide. 4-1BB can act as a ligand to tumor necrosis factor (TNF) and has stimulatory activity. 4-1BBL is covalently connect to the 5′ terminal of the extracellular antigen binding domain or, to the 3′ terminal of the intracellular domain.

In particular embodiments of this application, 4-1BB polypeptide is selected from a polypeptide with a sequence of amino acids shown by SEQ ID NO:12.

In particular embodiments, 4-1BB polypeptide has a continuous amino acid sequence of SEQ ID NO:12 showed, and its length is at least 20, or at least 30, or at least 40, or at least 50, less and up to 255 amino acids.

In the third aspect, the application comprises a nucleic acid molecule of encoding the specific chimeric antigen receptor targeting the human CD47 described in second aspect, wherein the nucleic acid molecules comprise a nucleotide sequence encoding guiding sequence sequentially connected from 5′ to 3′ a nucleotide sequence encoding human SIRPα targeting the human CD47, a nucleotide sequences encoding the transmembrane domain, and a nucleotide sequence encoding intracellular signaling domain, wherein the polynucleotide encoding an extracellular recognition domain that binds to human CD47 can be modified by codon optimization. Codon optimization can alter naturally occurring and recombinant gene sequences to achieve the highest possible level of productivity in any given expression system. Factors involved in different stages of protein expression include codon adaptability, mRNA structure, and various cis-elements in transcription and translation.

In some embodiments, the nucleic acid molecules also comprise a nucleotide sequences encoding the hinge region.

In some embodiments, the intracellular signaling domain comprises a receptor tyrosine activating motif] and the costimulatory signaling domain;

In some exemplary embodiments, the nucleic acid molecules respectively comprise nucleotide sequence encoding a guiding sequence, and the human SIRPα sequence in the first aspect of this application, and human CD8 hinge region sequence, and human CD8 transmembrane region sequence, and human CD28 intracellular domain sequence, and human 4-1BB intracellular domain sequence and human CD3 zeta intracellular domain sequence from 5 ‘to 3’ sequentially.

In some exemplary embodiments, the nucleic acid molecules respectively comprise nucleotide sequence for encoding a guiding sequence, and the human SIRPα sequence in the first aspect of the application, and the human CD8 transmembrane region sequence, and the human 4-1BB intracellular domain sequence and the human CD3 zeta intracellular domain sequence from 5 ‘to 3’ sequentially.

In some exemplary embodiments, the nucleic acid molecules respectively comprise nucleotide sequence encoding a guiding sequence, and the human SIRPα sequence in the first aspect of this application, and human CD8 hinge region sequence, and human CD8 transmembrane region sequence, and human 4-1BB intracellular domain sequence and human CD3 zeta intracellular domain sequence from 5 ‘to 3’ sequentially.

In some embodiments, the transmembrane domain comprises a CD8 transmembrane domain, a CD28 transmembrane domain, a CD3 zeta transmembrane domain, a CD4 transmembrane domain, a 4-1BB transmembrane domain, an OX40 transmembrane domain, ICOS transmembrane domain, CTLA-4 transmembrane domain, PD-1 transmembrane domain, LAG-3 transmembrane domain, 2B4 transmembrane domain, BTLA transmembrane domain, synthetic peptide (not based on immune response) Any of the related proteins).

In some embodiments, the immunoreceptor tyrosine activation motif comprises an intracellular signal domain of a CD3 zeta chain or an FcεRI gamma intracellular signal structure.

In some embodiments, the costimulatory signal domain comprises at least one of a CD28 intracellular signaling domain, a CD137/4-1BB intracellular signaling domain, a CD134/OX40 intracellular signaling domain, and an ICOS intracellular signaling domain.

In some embodiments, the hinge region is selected from the group consisting of a CD8 alpha hinge region, an IgG hinge region, or a hinge region or hinge region comprising all or part of an immunoglobulin that has been modified by one or more amino acids.

In some embodiments, the nucleotide sequence encoding human SIRPα targeting the extracellular recognition domain of human CD47 is shown as SEQ ID NO:17 or has identity of 80%, 85%, 90%, 95% or 99% with the sequence shown by SEQ ID NO:17.

In an exemplary embodiment, the nucleotide sequence encoding human SIRPα targeting the extracellular recognition domain of human CD47 is shown as SEQ ID NO:18 or has the identity of 80%, 85%, 90%, 95% or 99% with the sequence shown by SEQ ID NO:18.

In an exemplary embodiment, the nucleotide sequence encoding human SIRPα targeting the extracellular recognition domain of human CD47 is shown as SEQ ID NO:19 or has the identity of 80%, 85%, 90%, 95% or 99% with the sequence shown by SEQ ID NO:19.

In one embodiment, the nucleotide sequence encoding human SIRPα targeting the extracellular recognition domain of human CD47 is shown as SEQ ID NO:20 or has the identity of 80%, 85%, 90%, 95% or 99% with the sequence shown by SEQ ID NO:20.

In one embodiment, the nucleotide sequence encoding human SIRPα targeting the extracellular recognition domain of human CD47 is shown as SEQ ID NO:21 or has the identity of 80%, 85%, 90%, 95% or 99% with the sequence shown by SEQ ID NO:21.

In one embodiment, the nucleotide sequence encoding human SIRPα targeting the extracellular recognition domain of human CD47 is shown as SEQ ID NO:22 or has the identity of 80%, 85%, 90%, 95% or 99% with the sequence shown by SEQ ID NO:22.

In some embodiments, the nucleotide sequence encoding human CD8 hinge region is shown as SEQ ID NO:23 or has the identity of 80%, 85%, 90%, 95% or 99% with the sequence shown by SEQ ID NO:23.

In some embodiments, the nucleotide sequence encoding human CD8 transmembrane region is shown as SEQ ID NO:24 or has the identity of 80%, 85%, 90%, 95% or 99% with the sequence shown by SEQ ID NO:24.

In some embodiments, the nucleotide sequence encoding human 4-1BB intracellular domain is shown as SEQ ID NO:25 or has the identity of 80%, 85%, 90%, 95% or 99% with the sequence shown by SEQ ID NO:25.

In some embodiments, the nucleotide sequence encoding CD28 intracellular domain is shown as SEQ ID NO:26 or has the identity s of 80%, 85%, 90%, 95% or 99% with the sequence shown by SEQ ID NO:26.

In some embodiments, the nucleotide sequence encoding CD3 zeta intracellular domain is shown as SEQ ID NO:27 or has the identity of 80%, 85%, 90%, 95% or 99% with the sequence shown by SEQ ID NO:27.

In some embodiments, the nucleotide sequence encoding CD3 zeta intracellular domain is shown as SEQ ID NO:28 or has the identity of 80%, 85%, 90%, 95% or 99% with the sequence shown by SEQ ID NO:28.

In the fourth aspect, this application provides the recombinant vector or expression plasmid comprising the chimeric antigen receptor in the second aspect or the nucleic acid in the third aspect of the application.

The gene modification to immune response cells (for example, T cells, CTL cells, NK cells) can be achieved by using recombinant DNA or RNA to construct cell transducers of basically homologous cell compositions. In one embodiment, the vector is retroviral vector (eg., gamma retrovirus or lentivirus), which can transduce DNA or RNA constructs into the host cell genome. For example, the polynucleotides encoding SIRPα specific CAR targeting the human CD47 can be cloned into a retroviral vector and can be expressed from its endogenous promoter, the long terminal repeat sequence of the retrovirus, or from an alternative internal promoter.

The non-viral vector or RNA or usomh Random chromosome integration or targeted integration (such as nuclease, transcriptional activation of TALEN, ZFN, and/or regular cluster interval short palindrome repetition (CRISPR) or transgenic expression (for example, using natural or chemically modified RNA) may be another alternative to construct the recombinant vector or expression plasmid.

In some embodiments, the vectors may be selected from the gamma retrovirus vector, lentiviral vector, adenovirus vector, or adenosine related virus vector. In an example embodiment, the vector is a gamma retroviral vector.

In the fifth aspect, the present application provides a promoter for constructing a recombinant vector in the fourth aspect of the application and expressing the specific chimeric antigen receptor targeting the human CD47 in the second aspect of the application, the promoters comprise, but are not limited to, nucleotide sequences, such as EF1 alpha promoter shown in SEQ ID NO:29 and EFS promoters as shown in SEQ ID NO:30.

In an exemplary preferred embodiment, the promoter is used to construct the recombinant vector in the fourth aspect and to express the specific chimeric antigen receptor targeting the human CD47 in the second aspect is the EF1 alpha promoter as shown by the SEQ ID NO:29.

In an exemplary preferred embodiment, the promoter is used to construct the recombinant vector in the fourth aspect and to express the specific chimeric antigen receptor targeting the human CD47 in the second aspect is the EFS promoter as shown by the SEQ ID NO:30.

In the sixth aspect, the present application provides a recombinant virus, which can express chimeric antigen receptor targeting to CD47 and infect the immune response cells in the second aspect of the invention.

In some embodiments, the immune response cells are cytotoxic T lymphocytes, NK cells, and NKT cell or auxiliary T cells, etc.

In an exemplary embodiment, the immune response cells are cytotoxic T lymphocytes.

In some embodiments, the virus is lentivirus, adenovirus, adenosine related virus or retrovirus.

In an exemplary embodiment, the virus is lentivirus.

In an exemplary embodiment, the virus is adenovirus.

In the seventh aspect, the present application provides an isolated modified immune response cell comprising the chimeric antigen receptor in the second application, which is derived in the transformants from the recombinant vector or an expression plasmid in the third aspect of the application.

In respect to the initial gene modification of the cell to provide targeting the human CD47 specific immune response cells, retroviral vectors are usually used in transduction, however any other appropriate viral vector or non viral delivery system may be used. In respect to the subsequent gene modification to provide cells comprising at least two kinds of antigen presenting complexes with costimulatory ligands, retroviral gene transfer (transduction) is also proved to be effective for cells. The combination of retroviral vectors and suitable assembly lines is also suitable, in which capsid proteins are functional for human infected cells.

In some embodiments, the immune response cell comprises at least one exogenous costimulatory ligand.

Optional transduction methods also comprise direct co-culture of cells and production cells.
In some embodiments, a possible transduction method further comprises direct co-cultivation of the cells with the producer cells. The viral vectors for transducing can be used to express costimulatory ligands (e.g., 4-1BBL and IL-12) in immune response cells. Preferably, the selected vector exhibits high infection efficiency and stable integration and expression.

In some embodiments, preferably, the at least one of the costimulatory ligand is selected from 4-1BBL, CD80, CD86, CD70, OX40L, CD48, TNFRSF14 and their composition, or more preferably, the costimulatory ligand is 4-1BBL.

In some embodiments, the immune response cell is selected from the T cells, natural killer (NK) cells, cytotoxic T lymphocytes (CTL), regulatory T cells, human embryonic stem cells and pluripotent stem cells that differentiate into lymphoid cells, preferably T cells and natural killer (NK) cells, preferable as the T cells.

In some embodiments, vectors for CAR expression may be used to transduce multiple T cell subsets isolated from patients.

The seventh aspect of this application disclose isolated and modified immune response cells, which express the extracellular identification domain able to specifically targeting the human CD47, and which are used to treat or prevent tumor formation, immune disease, or for antiaging.

In an exemplary embodiment, wherein the isolated and modified immune response cells are CAR-T cells.

The genetically modified central memory type T cells could be prepared from human SIRPα chimeric antigen receptor targeting the human CD47 and then kept in cold storage.

In the eighth aspect of the present application provides a method for preparing an isolated and modified chimeric antigen receptor modified immune response cell in the sixth aspect of this application, include the following steps:

First, ligates and isolated the nucleic acid molecules in the third aspect into the expression vector through molecular cloning, and acquired the specific expression vector of the specific chimeric antigen receptor targeting to CD47. Then, transduced the specific CAR expression vector into 293T cells to acquire the virus solution. Finally, used the virus to infect the immune response cells, and produced the CAR-T cells expressing the specific chimeric antigen receptor targeting to CD47 after virus infection.

In some particular embodiments, the immune response cells modified in the invention can be cells of lymphoid lineages. The cells of the lymphatic family are selected from the B, T and natural killer (NK) cells, which provide the production of antibodies, the regulation of the cellular immune system, the detection of foreign substances in the blood, and the function detection foreign cells of the host. Lymphoid cell lines in particular embodiments comprise the T cells, natural killer (NK) cells, cytotoxic T lymphocytes (CTL), regulatory T cells, embryonic stem cells, and pluripotent stem cells (for example, pluripotent stem cells that can be differentiated into lymphoid cells).

In some embodiments, the immune response cells are selected from the T cells, natural killer (NK) cells, cytotoxic T lymphocytes (CTL), regulatory T cells, human embryonic stem cells, and pluripotent stem cells which could be differentiated into the lymphoid cells, preferring T cells or natural killer (NK) cells. In some instance embodiments, T cells are mature lymphocytes in the thymus and mainly response for cell-mediated immunity. T cells participate in the acquired immune system.

In some particular embodiments, T cells comprise but are not limited to helper T cells, cytotoxic T cells, and memory T cells (including central memory T cells, stem like memory T cells (or stem like memory T cells) and two types of effector memory T cells (e.g., TEM and TEMRA cells), regulatory T cells (also known as inhibitory T cells), natural killer cells, sticky membrane related constant T cells, and gamma delta T cells. In some embodiments, T cells expressing CAR express Foxp3 to achieve and maintain T regulatory phenotype.

The CAR-T cells in this application can comprise at least one exogenous costimulatory ligand, which makes the immune response cells co-expressing exogenously or be induced to co-express exogenously based on SIRPα, specific chimeric receptor targeting the human CD47 and at least one exogenous costimulatory ligand. The interaction between the specific CAR targeting the human CD47 and at least one of the costimulatory ligands provides an important non antigen specific signal for the complete activation of immune response cells, such as T cells. In some embodiments, at least one costimulatory ligand is selected from 4-1BBL, CD80, CD86, CD70, OX40L, and composition thereof. In one embodiment, the costimulatory ligand is 4-1BBL. In one embodiment, the costimulatory ligand is 4-1BBL.

In a preferred embodiment, the isolated modified immune response cells are T cells.

In a preferred embodiment, the isolated modified immune response cells are natural killer (NK) cells

In some particular embodiments, the isolated and modified immune response cells (such as T cells) may be autologous, non autologous (for example, allogeneic), or derived from engineered progenitor cells or stem cells in vitro.

In the ninth aspect, the application provides a pharmaceutical composition, which comprises the effective dose of the CAR-T cells in the sixth aspect of the invention and pharmaceutical acceptable excipients.

The disclosed drug composition in the application comprises the CAR-T cells and a pharmaceutically acceptable vector that expresses the specific chimeric antigen receptor targeting the human CD47.

The application of the pharmaceutical composition can be autologous or allogeneic. For example, the immune response cells expressing the specific CAR cells targeting the human CD47 and the composition can be acquired from one subject and applied to the same subject or different compatible subjects. Local injection, including catheter delivery, whole body injection, local injection, intravenous injection, or gastrointestinal administration, can be used to use T cells or their progeny of the peripheral blood of the invention (for example, in vivo, in vitro, or in vitro derived). The public subject pharmaceutical composition in this invention is applied such as a pharmaceutical composition comprising an immune response cell expressing SIRPα specific CAR, is used, it is usually prepared into a unit dose injectable form as solution, suspension, emulsion.

The composition of the application can be the preparation, the immune response cells express the specific chimeric antigen receptor (CAR) targeting the human CD47 and the composition thereof, which can be conveniently provided as a sterile liquid preparation, such as a water solution, a suspension, a emulsion, a dispersive solution or a sticky composition, which can be buffered to the selected liquid formulations are usually easily prepared than gel, other viscous compositions and solid compositions. In addition, liquid compositions are more convenient to use, especially by injection. On the other hand, the composition of stickiness samples can be prepared in the proper viscosity range to provide longer contact time with specific tissues. Liquid or a viscous composition may comprise a carrier, which may comprise water, saline, phosphate buffer salts. Solvents or fractions of water, polyols (e.g. glycerol, propylene glycol, liquid polyethylene glycol, etc.), suitable mixtures and dispersive medium thereof.

The additives, including antimicrobial, preservatives, antioxidants, chelating agents and buffers, can be added to enhance the stability and aseptic properties of the composition.

All of carriers, diluents or additives used in this application must be compatible with an immune response cell expressing a specific chimeric antigen receptor (CAR) targeting the human CD47 in the public theme of the invention.

If necessary, the viscosity of the composition can be maintained at a selected level using a pharmaceutically acceptable thickening reagents. The selection of appropriate carriers and other additives depend on the exact approach of delivery and the nature of specific dosage forms, such as liquid dosage forms (for example, whether the composition is prepared in a solution, a suspension, a gel or another liquid form, such as a time release form or a liquid filling form).

In the tenth aspect, the present application provides a kit for the treatment or prevention of tumor formation, pathogen infection, autoimmune disease, allograft, or graft rejection or anti-aging, wherein comprises the immune response cells in a sixth aspect or the nucleate in a third aspect of the invention of the invention.

If necessary, the immune response cells are provided with the instructions together, which are applied to the subjects with symptom of a risk of tumor formation, or pathogen infection, or immune disease, or allograft or senescence. Instructions usually comprise information about compositions used in the treatment or prevention of tumor formation, pathogen infection, immune diseases or allograft. In other embodiments, the instructions comprise at least one of the following: a description of a therapeutic preparation which comprises a dose regimen and application method for the treatment or prevention of the formation of a tumor, a pathogen infection, an immune disease or a allograft or its symptoms; note which comprises taboo; indications; non indication; information overload; adverse reactions; animal pharmacology; clinical research; and/or reference. The instructions can be directly attached on the container (when it exists), or as a label on the container, or as a separate page, handbook, card, or folding print, in the container or with the container.

In the eleventh aspect, the application provides human SIRPα protein and its variants targeting the human CD47 in the first aspect of the invention, and the chimeric antigen receptor targeting the human CD47 in the second aspect, and the recombinant vector or expressing plasmid in the fourth aspect, and the recombinant virus in the fifth aspect, and the isolated and modified immune response cells are acquired by preparation method in the seventh aspect, and composition in the eighth aspect, and the application of kits in the treatment or prevention of diseases in the ninth aspect, which the treatment or prevention comprises the application of the effective dose of SIRPα targeting to CD47 CAR-T cells in the second aspect of this application to the patients with diseases caused by specific interactions with CD47 and SIRPα proteins.

In some embodiments, the diseases comprises tumor formation, pathogen infection, autoimmune diseases, allograft, graft rejection, and aging.

In some embodiments, the tumor formation, infection of pathogens, autoimmune diseases, inflammatory diseases, allograft, or transplant rejection or senescence are related to the specific interaction of CD47 and SIRPα proteins.

In some embodiments, the treatment or prevention of tumor formation involves increasing the growth of immune activated cytokines by reducing the tumor load in the subjects, increasing or prolonging the survival of the subjects with the tumor formation or responding to the cancer cells or pathogens of the subjects.

In some embodiments, tumor or tumor formation is selected from the glioblastoma, liver cancer, pancreatic cancer, acute myeloid leukemia, colorectal cancer, medulloblastoma, gastric cancer, ovarian cancer, lung cancer, prostate cancer, breast cancer and neuroblastoma, bladder cancer, colon cancer, renal cell carcinoma, leukemia, lymphoma, multiple myeloma, melanoma, and its composition (and to correspond target cells tested in embodiments).

In an exemplary embodiment, the tumor or tumor formation is glioblastoma.

In an exemplary embodiment, the tumor or tumor formation is medulloblastoma.

In an exemplary embodiment, the tumor or tumor formation is acute myeloid leukemia.

In an exemplary embodiment, the tumor or tumor formation is liver cancer.

In an exemplary embodiment, the tumor or tumor formation is pancreatic cancer.

In an exemplary embodiment, the tumor or tumor formation is colorectal cancer.

The invention is based on the human SIRPα molecule to construct specific chimeric antigen receptor (CAR) targeting the human CD47 and the CAR modified T cells (CAR-T cell). The new CAR-T cells can effectively target a variety of tumor cells and can be developed to the preparations for the treatment of tumors, especially the CD47 positive expression tumor.

In the study, the inventor of the invention has unexpectedly discovered that the preparation method is convenient of the isolated T cells which are modified by human SIRPα chimeric antigen receptor, wherein the effector to target ratio is 5:1, the killing ratio of tumor cells is from 50% to 90%, and also can significantly prolong persistence of the immune cells in patients, and improve the immune cells specifically targeting to tumor cells, especially those CD47 positive tumors, and improve the immune cells cytotoxicity for glioblastoma, and wherein the modified immune response cells that express human chimeric SIRPα receptor targeting to human CD47 positive tumors are described in the invention, which is new approach option for tumor immunotherapy, and has potential industrial application prospects.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

The following is further explained by the embodiment, but the invention is not limited to these specific Examples.

The materials and reagents used in the following embodiments can be acquired from commercial companies without special explanation.

### Example 1

Preparation of Expression Plasmid which Expresses Specific Chimeric Antigen Receptor Targeting to CD47.

Step 1), determination of amino acid sequence of specific chimeric antigen receptor targeting to CD47.

First, full length ORF amino acid sequences (as shown by SEQ ID NO:1) and full length encoding nucleotide sequences (as shown by SEQ by ID NO:2) are searched and found from the NCBI Genbank database of the National Medical Library of the United States.

Second, construction of the specific chimeric antigen receptor targeting to CD47 as follows.

The amino acid sequences of specific CAR molecules targeting to CD47 from the amino terminal to the carboxyl end, which are sequentially formed with the amino acid sequence of the guided peptide (as shown by SEQ ID NO:3), and the amino acids sequence of the human SIRPα(as shown by SEQ ID NO:4), and the amino acid sequence of the human CD8 hinge region (as shown by SEQ ID NO:10), and the amino acid sequence of human CD8 transmembrane region (as shown by SEQ ID NO:11), and the amino acid sequence of the intracellular domain of human 4-1BB (as shown by SEQ ID NO:12), and the amino acid sequence of the human CD3 zeta domain (as shown by SEQ ID NO:14) and sequentially connected.

The nucleotide sequence of specific CAR molecules targeting to CD47 from the 5′ terminal to the 3′ terminal, which is sequentially connected with nucleotide sequence of the encoding guide sequence (as shown by SEQ ID NO:16), and nucleotide sequence encoding the human SIRPα(as shown by SEQ ID NO:17), and nucleotide sequence encoding the human CD8 hinge region (as shown by SEQ ID NO:23), and nucleotide sequence encoding the human CD8 transmembrane region (as shown by SEQ ID NO:24), and nucleotide sequence encoding the intracellular domain of human 4-1BB (as shown by SEQ ID NO:25), and nucleotide sequence encoding the human CD3 zeta domain (as shown by SEQ ID NO:27).

Step 2), construction and identification of plasmid expressing the specific CAR molecule targeting to CD47.

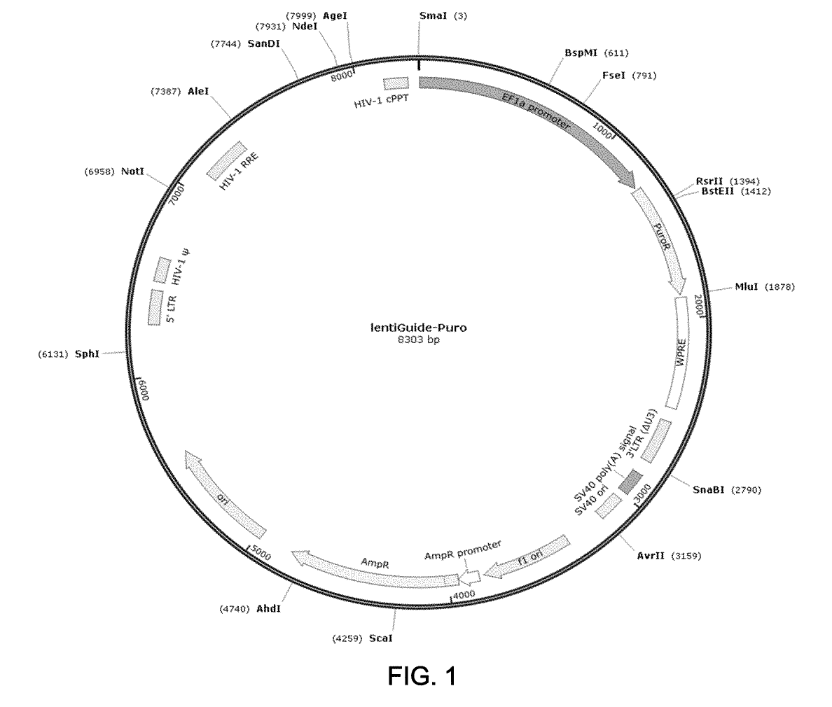

The nucleotide sequences of the specific CAR molecules targeting to CD47 by de novo synthesis are connected to the lentivirus vector lentiGuide-Puro (Addgene as shown by FIG. 1 shown) by molecular cloning, and the full length CAR sequence expression frame of a single coding frame is constructed and expressed by using the EF1 alpha promoter (described in sequence table as shown by SEQ ID NO:29). The specific operation steps are as follows.

De novo synthesize the nucleotide sequence of the specific CAR molecules targeting to CD47 comprises the guiding sequence, and human SIRPα sequence, and CD8 hinge region, and CD8 transmembrane region, and 4-1BB intracellular domain, and CD3 zeta domain (Idobio, Nanjing), which was amplified by PCR with primers as follow showed:

5′-cactttggcgccggctcgagggggcccgggtgcaaagatggataaagttttaaacagagagga-3′ (described in sequence table as shown by SEQ ID NO:31) or 5′-cactttggcgccggctcgagggggcccgggtaggtcttgaaaggagtgggaattggctcc-3′ (described in sequence table as shown by SEQ ID NO:32) and
5′-tccagaggttgattgtcgacttaacgcgtttagcgagggggcagggcctgcatgtgaag-3′ (described in sequence table as shown by SEQ ID NO:33) and recovered by Axygen gel Recovery Kit (ZeHeng, Hangzhou), and then the homologous recombination ligation was performed with the lentiGuide-Puro vector (Addgene), which was digested by the restriction endonuclease SmaI and MluI. The system and conditions for the particular recombination ligation reaction as follows.

Recombination ligation system:

ligation reaction system comprises the 5 μl recovered PCR products by gel purification, and the 3 μl plasmid which was digested by the restriction endonuclease SmaI and MluI by gel purification, and 5 μl of 4×1402 QuickCloning Kit (Jinuomei, Nanjing) and 7 μl ddH2O with total volume of 20 μl.

Recombination ligation condition: the above reaction system was placed in 50 degree Celsius water bath, which was placed on the ice 1 min after water bath 15 mins.

The recombinant products of 10 μl were transformed into the competent Stbl3, and the specific transformation steps were as follows.

The 5 μl product was added to 50 μl of Stbl3 competent cells (purchased from Invitrogen), and then ice bath 30 min, heat shock at 42° C. 45s, ice bath 2 min again, and then add 500 μl without LB liquid medium, 37° C. 200 rpm vortex culture 40 min, inoculated cells into ampicillin resistant LB solid plate, cultured overnight in shaking incubator at 37° C. After single colony emerged, pick up 5 moderately sized colonies, extract plasmids and send out for sequencing (Idobio, Nanjing). Sequenced the company sequencing (Idobio, Nanjing), sequenced the results and synthesized the SIRPα specific CAR. Comparing the sequencing results with the sequence of the synthesized SIRPα specific CAR molecule, the sequence was completely correct and proved that the expression plasmids of the specific CAR targeting to CD47 was obtained (Abbreviation of KD-045 CAR lentivirus vector, EFS promoter).

Step 3), extraction and purification of specific CAR expression plasmid targeting to CD47.

In step 2) the Stbl3 strain comprising the plasmid that expresses the specific CAR molecule targeting to CD47 is cultured in LB medium, and is extracted by using the high purity and without endotoxin Qiagen Plasmid Midi Kit (Qiagen, German) for the infecting purpose. The operation steps thereof are as follows.

1. Harvest overnight bacterial culture 150 ml in a centrifuge tube by centrifuging at 6000× g for 15 mins, as much as possible to remove the supernatant (bacterial cells can be precipitated by multiple centrifugation to collect into one centrifuge tube if the bacterial liquid is overload).

2. Completely suspend the bacterial pellet in 4 ml buffer P1 at the centrifuge tube by a pipette or a vortex oscillator (check if RNase A was added first).

3. Add 4 ml buffer P2, mix thoroughly by softly inverting 4-6 times, and incubate at room temperature for 5 mins.

4. Add 4 ml prechilled buffer P3, mix thoroughly by softly inverting 4-6 times, and incubate at room temperature for 5 mins.

5. Centrifuge at 20000× g for 10 mins at 4° C.

6. Equilibrate a QIAGEN-tip 100 by applying 4 ml buffer QBT, and allow column to empty by gravity flow.

7. Apply the supernatant from step 5 to the QIAGEN-tip and allow it to enter the resin by gravity flow.

8. Wash the QIAGEN-tip with 2×10 ml buffer QC which is allowed to move through the QIAGEN-tip by gravity flow, repeat operation again.

9. Add 5 ml elution buffer QF into the QIAGEN-tip and collect it into a 15 ml new centrifuge tube.

10. Precipitate DNA by adding 3.5 ml room-temperature isopropanol to the eluted DNA and mix thoroughly, and centrifuge at 15000 for 30 mins at 4° C., and carefully decant the supernatant.

11. Wash the DNA pellet with 2 ml room-temperature 70% ethanol and centrifuge at 15000 for 30 mins at 4° C., and carefully decant the supernatant.

12. Air-dry pellet for 5-10 mins and redissolve DNA in a suitable volume of appropriate buffer, and then completely transfer DNA solution into a new 1.5 ml centrifuge tube.

### Example 2

Preparation of the Plasmid that Expresses the Specific Chimeric Antigen Receptor Targeting to CD47 in the Embodiment 2.

In addition to the human SIRPα sequence in the amino acid sequence of the specific CAR molecule targeting to CD47 (as shown by SEQ ID NO:5), and the amino acid sequence of the CD3 zeta domain (as shown by SEQ ID NO:15), encodes the nucleotide sequence of human SIRPα in the nucleotide sequence of the specificity CAR molecules targeting to CD47 (as shown by SEQ ID NO:18), and step 2) the construction and identification of the specific CAR molecules of the targeting to CD47 and the identification of the nucleotide sequences of the specific CAR molecules targeting to CD47 by de novo synthesis, and then connected to the vector lentiGuide-Puro (Addgene, FIG. 1) by molecular cloning, and construct a full length CAR sequence of a single encoding frame by the EFS promoter (as shown by sequence table SEQ ID NO:30) in step 1, the rest is the same as the example 1.

### Example 3

Preparation of the Plasmid that Expresses the Specific Chimeric Antigen Receptor Targeting to CD47.

In addition to the human SIRPα sequence in the amino acid sequence of the specific CAR molecule targeting to CD47 (as shown by SEQ ID NO:6), and encodes the nucleotide sequence of human SIRPα in the nucleotide sequence of the specificity CAR molecules targeting to CD47 (as shown by SEQ ID NO:19) in step 1, the rest is the same as the example 1.

### Example 4

Preparation of the Plasmid that Expresses the Specific Chimeric Antigen Receptor Targeting to CD47.

In addition to the human SIRPα sequence in the amino acid sequence of the specific CAR molecule targeting to CD47 (as shown by SEQ ID NO:7), and encodes the nucleotide sequence of human SIRPα in the nucleotide sequence of the specificity CAR molecules targeting to CD47 (as shown by SEQ ID NO:20) in step 1, the rest is the same as the example 1.

### Example 5

Preparation of the Plasmid that Expresses the Specific Chimeric Antigen Receptor Targeting to CD47.

In addition to the human SIRPα sequence in the amino acid sequence of the specific CAR molecule targeting to CD47 (as shown by SEQ ID NO:8), and encodes the nucleotide sequence of human SIRPα in the nucleotide sequence of the specificity CAR molecules targeting to CD47 (as shown by SEQ ID NO:21) in step 1, the rest is the same as the example 1.

### Example 6

Preparation of the Plasmid that Expresses the Specific Chimeric Antigen Receptor Targeting to CD47.

In addition to the human SIRPα sequence in the amino acid sequence of the specific CAR molecule targeting to CD47 (as shown by SEQ ID NO:9), and encodes the nucleotide sequence of human SIRPα in the nucleotide sequence of the specificity CAR molecules targeting to CD47 (as shown by SEQ ID NO:22) in step 1, the rest is the same as the example 1.

### Example 7

Isolation and Culture of T Cells.

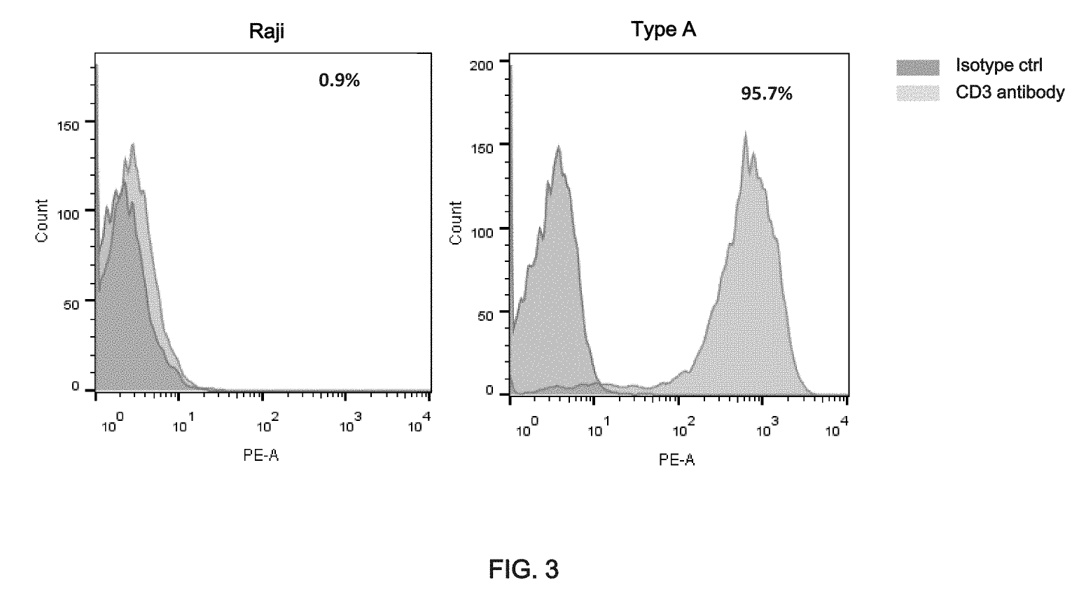

Fresh peripheral blood mononuclear cells were isolated from the fresh peripheral blood of healthy donors by density gradient centrifugation, and then used paramagnetic beads coupled with anti-CD3 antibodies and anti-CD28 antibodies (purchased from Invitrogen, USA; Dynabeads Human T-Activator CD3/CD28, catalogue number: 11161D described as the product information) to enrich CD3+T cells. Specifically, peripheral blood mononuclear cells were diluted to the concentration of (10˜30)×10⁶ cells/ml, then the magnetic beads and cells were mixed thoroughly in the ratio of 3:1 and incubated culture dishes for 2˜3 hours at room temperature, and then enriched CD3+T cells by using magnetic particle collector (Magnetic particles Concentrator, referred to as MPC, purchased from Invitrogen, USA, catalogue number: 12301D). Finally, the enriched CD3+T cells were suspended in the culture medium (purchased from Life Technologies, USA, pTmizerT-Cell Expansion SFM, A1048503 described as product information), and adjusted to the solubility at 1×10⁶ cells/ml, continue to culture at 37° C. and 5% CO2 incubator for 2 days. T cell purity was assayed by using flow cytometry with anti PE anti-human CD3 antibody (purchased from BioLegend, USA, catalogue number: 300408), which the results showed that the purity of T cells after magnetic bead enrichment was more than 95% (FIG. 3).

### Example 8

Preparation of Virus Solution.

Step 3 of embodiment 1) acquired the plasmid that expressed the specific CAR targeting to CD47, and the package plasmids psPAX2 and VSVG, which were mixed at the ratio of 10:8:5, and co-transfected into 293T cells (ATCC product, product number: CRL-3216) by using the polyethylene imide transfection reagent (408727, Sigma). The preparation method of package plasmid is referred to Lenti-X Packaging Single Shots instructions (Takara), and the specific transfection procedure is referred to Sigma transfection manual.

After 6 hours transfection, the medium was replaced with a complete medium (purchased from Life Technologies, product number: 11995-065). After 48 hours and 72 hours culture, the virus supernatant was collected respectively and centrifuge at 3000 rpm for 10-15 mins at 4° C. and then was filtered by 0.45 micron membrane. Finally, Super-centrifuge at 25000 rpm for 2˜3 hours at 4° C., and then, the virus was condensed. After that the virus was transferred to −80° C. for storage.

### Example 9

Preparation of Specific CAR-T Cells Targeting to CD47.

The CD3+T cells acquired from embodiment 2 were inoculated into 24 well plates with the concentration of 1×10⁵ cells/ml, and then cultured for 24 hours at 37° C. and 5% CO2 (culture time based on practice. Typically, the cell confluence rate was between 50%-70% when the virus infected).

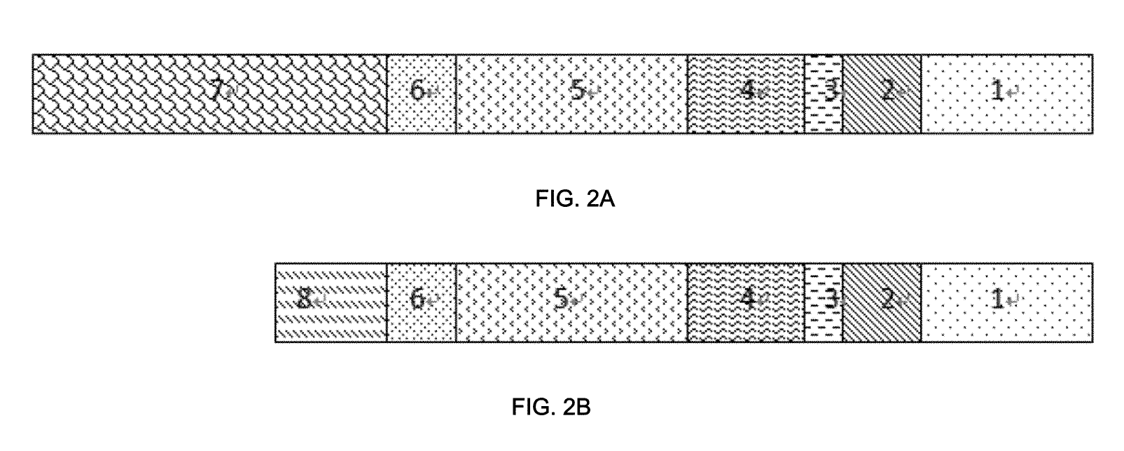

Next day, taking the virus concentrate from embodiment 3, which was added to the cell culture bottle at the value of MOI=1˜10. Sealed and centrifuged at low speed (500 g-1000 g/min) for 30˜60 minutes, and then placed it to culture at 37° C. incubator. After 48 hours infection, the T cell KD-045 CAR-T cell expressing SIRPα CAR molecule, namely the new CAR-T cell, was obtained, and the molecular structure of CAR was shown in FIGS. 2A and 2B, which can be performed the next functional experiment.

### Example 10: Identification

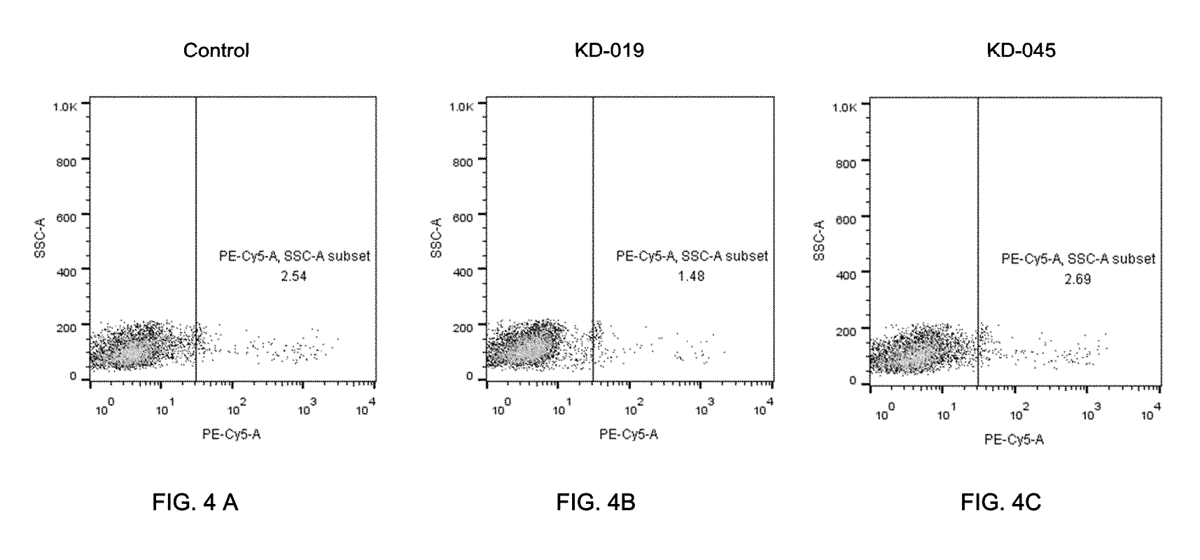

Then, the 7-AAD/CFSE cytotoxicity test kit (purchased from Biovision, catalogue number: K315-100) was used to test the cell activity of the prepared CAR-T cells according to the operation instructions of the kit. Flow cytometry data showed that the activity of KD-045 CAR-T cells was more than 97% (FIGS. 4A, 4B and 4C).

And then, the expression of CAR after infection was detected by flow cytometry.

Blank control group: T cells without virus infection.

KD-019 control group: specific CAR-T cells targeting to CD19,

Group KD-045: specific CAR-T cells targeting to CD47.

The detecting cells to be collected during the preparation of cells.

After washing twice with PBS, the above experimental group and the control group were suspended in FACS solution (comprising 0.1% sodium azide and 0.4% BSA PBS).

According to the antibody instructions, the anti-human SIRPα antibody (PE anti-human-CD172a, 320806, Biolegend) labeled with PE was added to the cell suspension of control group and the cell group detected, and incubated at 4° C. for 60 mins. Flow cytometry (BD FacsCanto II) was used to count the stained cells and then FlowJo software was used to analyze the results.

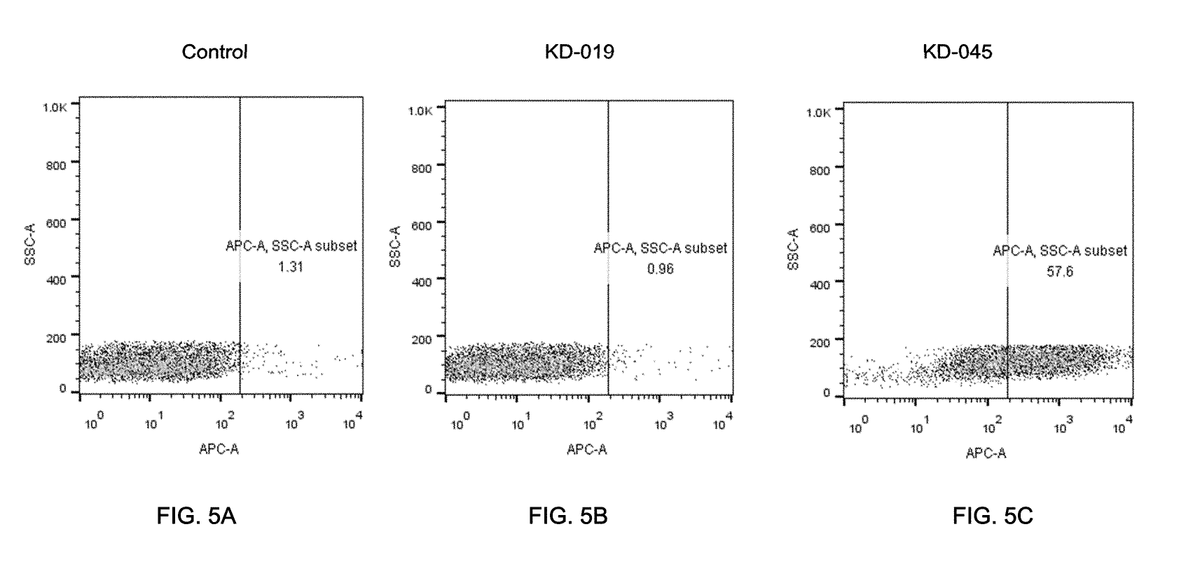

The results from flow cytometry as FIGS. 5A, 5B and 5C showed, which proved that the cells to be detected in the collection of embodiment 4 expressing the specific chimeric antigen receptor targeting to CD47.

Application examples for effect data thereof

The tumor cell line (also known as target cell line): glioblastoma U251 cell (purchased from cells resource center the Shanghai Institutes of life sciences, Chinese academy of sciences), medulloblastoma cell HTB186 (purchased from Jennio, Guangzhou), acute myeloid leukemia cell U937 (purchased from Jennio, Guangzhou), hepatoma cell SMMC7721 (purchased from Cobioer, Nanjing), pancreatic cancer cell BXPC3 and colorectal cancer cell HCT 116 (purchased from Jennio, Guangzhou).

First, we investigated the expression of CD47 in various target tumor cell lines referring to the previous research report (Murata et al (2018) Cancer Sci 109:2349-2357).

Next, by using the 7-AAD/CFSE cytotoxicity test kit (purchased from Biovision, catalogue number: K315-100), and according to the operation instructions of the kit to evaluate the killing of specific CAR-T cells targeting to the CD47 positive target cell line in the example 4.

Specifically, each target cell line was stained by CSFE fluorescence and was seeded in the culture plate at a concentration of 2×10⁴ cells/ml per well. One experiment group and two control groups were set up for each target cell line, in which the experimental group added the cell suspension of specific CAR-T cells targeting to CD47 whereas the blank control group added T cells that was CD3+T cells acquired embodiment was not infected by the virus, and the KD-019 control group added unrelated CAR-T cells targeting to CD19.

In the experimental group, three specific effector to target ratios (10:1, 5:1 and 1:1) were applied to mix specific CAR-T cells targeting to CD47, which targets CD47, and target cells. Here, the term “effector to target ratio” refers to the ratio of effector cells (targeting to CD47 specific CAR-T cells) to target cells (tumor cells).

Also, the blank control group and the KD-019 control group also mixed T cells with target cells according to three different effector to target ratios.

After culture for 20 hours, the centrifuge went to remove the supernatant. After cleaning the cell precipitates that were stained by 7AAD. Flow cytometry (BD FacsCanto II) was used to count the staining cells, and the results were analyzed by FlowJo software.

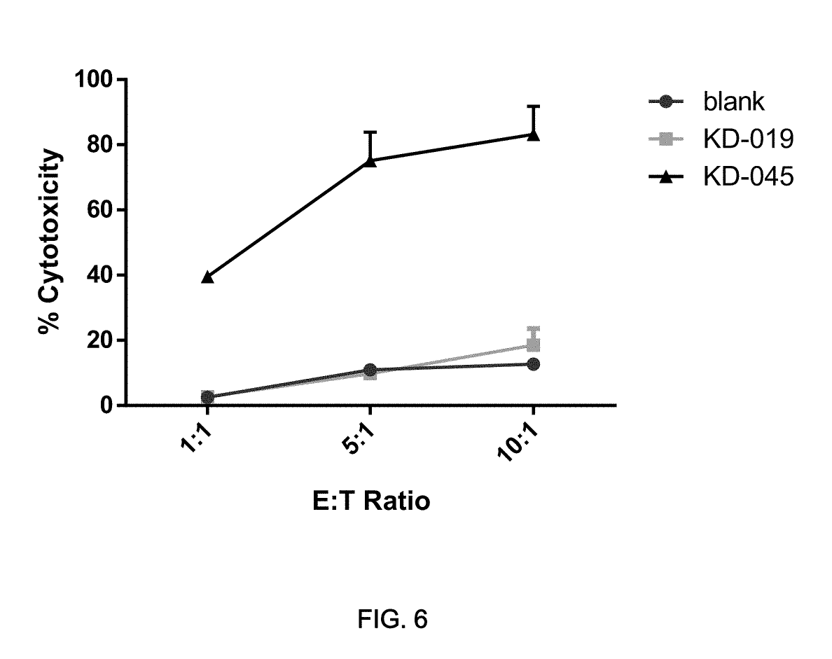

FIG. 6 shows the test results of tumor cell killing rate with glioblastoma cell U251 as target cell. As can be seen from FIG. 6, the specific CAR-T cell KD-045 targeting to CD47 in embodiment 4 has a significant killing effect on the glioblastoma cell U251 (obviously higher than the two control groups), and the tumor killing rate is over 80% at the ratio of effector to target 5:1.

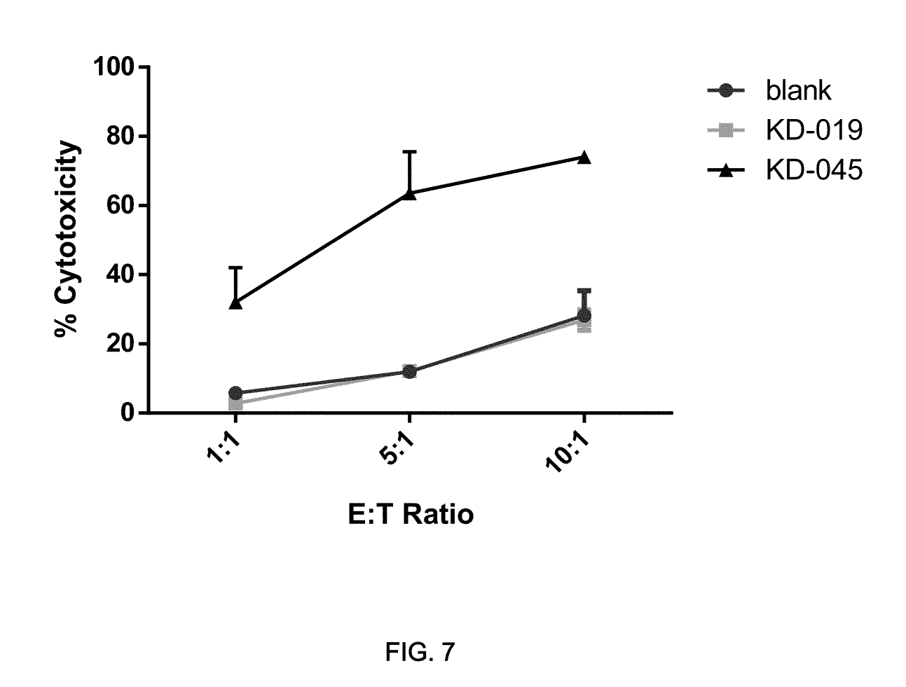

FIG. 7 shows the test results of tumor cell killing rate with medulloblastoma cell HTB186 as target cell. As can be seen from FIG. 7, the specific CAR-T cell KD-045 targeting to CD47 in embodiment 4 has a significant killing effect on the medulloblastoma cell HTB186 (obviously higher than the two control groups), and the tumor killing rate is over 60% at the ratio of effector to target 5:1.

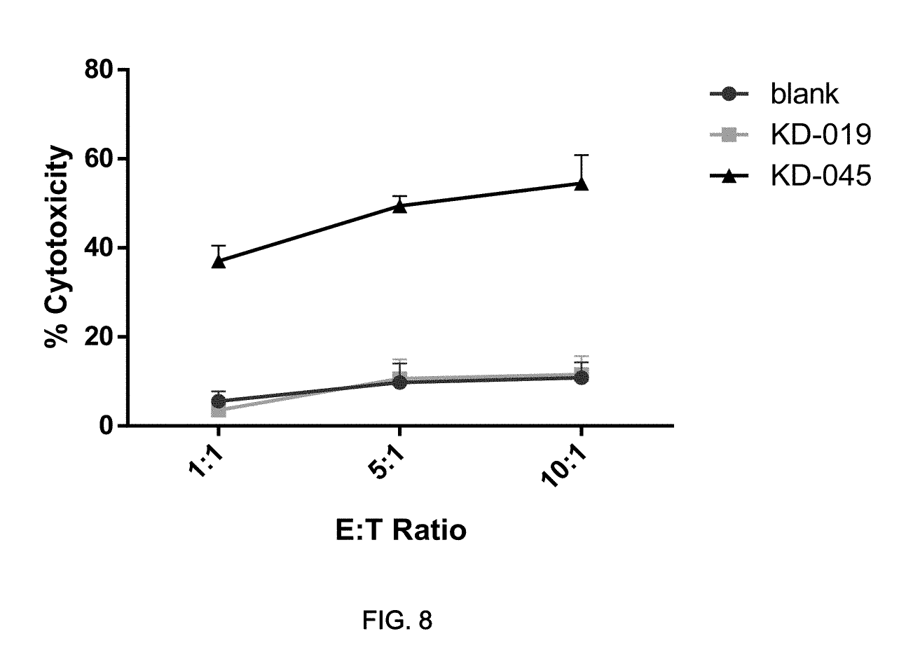

FIG. 8 shows the test results of tumor cell killing rate with acute myeloid leukemia cell U937 as target cell. As can be seen from FIG. 8, the specific CAR-T cell KD-045 targeting to CD47 in embodiment 4 has a significant killing effect on the acute myeloid leukemia cell U937 (obviously higher than the two control groups), and the tumor killing rate is over 50% at the ratio of effector to target 5:1.

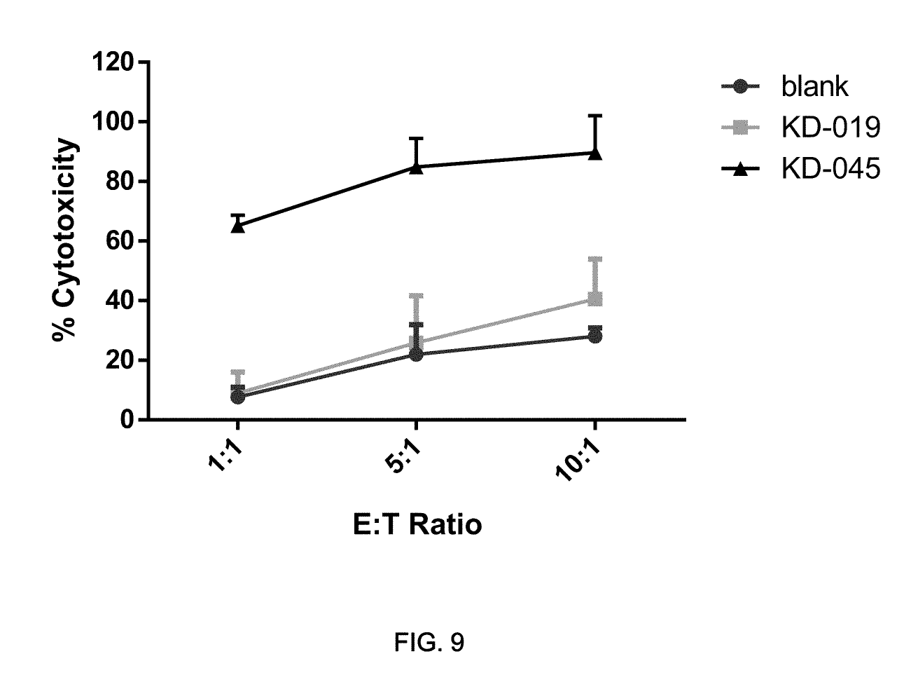

FIG. 9 shows the test results of tumor cell killing rate with hepatoma cell SMMC7721 as target cell. As can be seen from FIG. 9, the specific CAR-T cell KD-045 targeting to CD47 in embodiment 4 has a significant killing effect on the hepatoma cell SMMC7721 (obviously higher than the two control groups), and the tumor killing rate is over 85% at the ratio of effector to target 5:1.

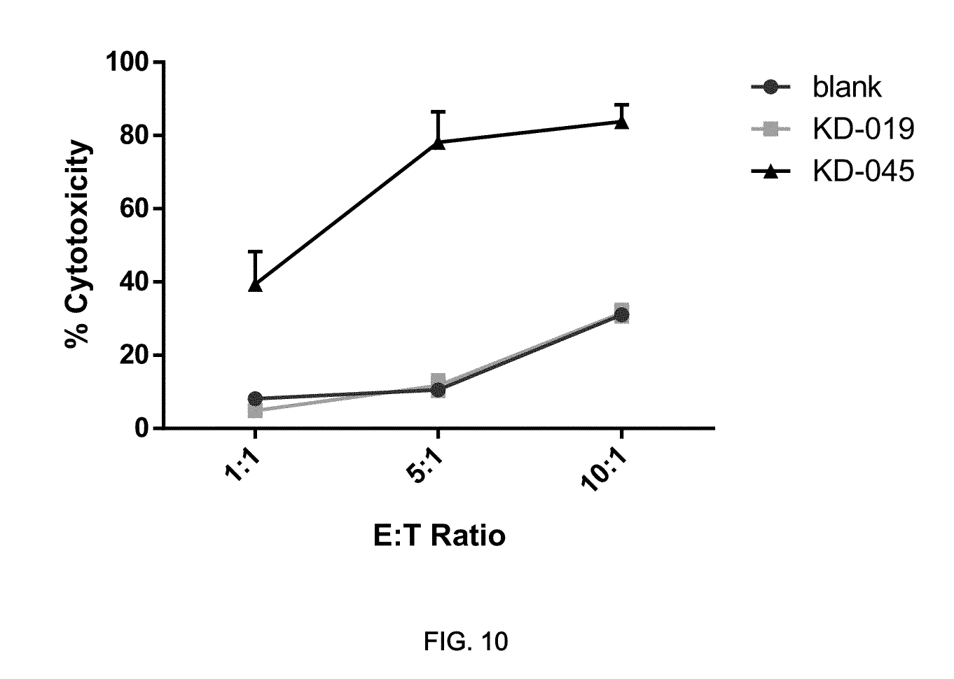

FIG. 10 shows the test results of tumor cell killing rate with pancreatic cancer cell BXPC3 as target cell. As can be seen from FIG. 10, the specific CAR-T cell KD-045 targeting to CD47 in embodiment 4 has a significant killing effect on the pancreatic cancer cell BXPC3 (obviously higher than the two control groups), and the tumor killing rate is over 80% at the ratio of effector to target 5:1.

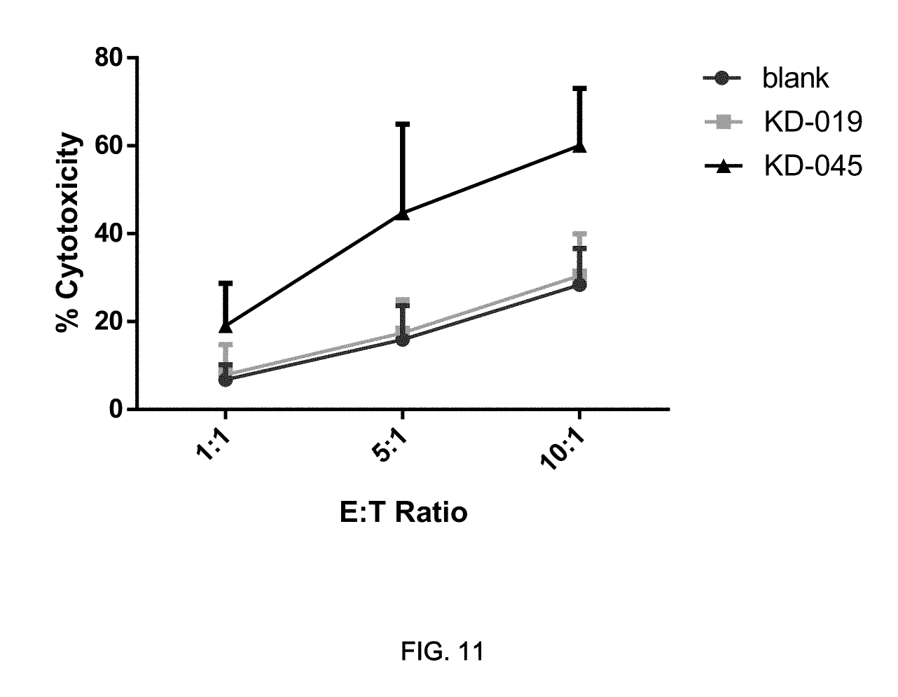

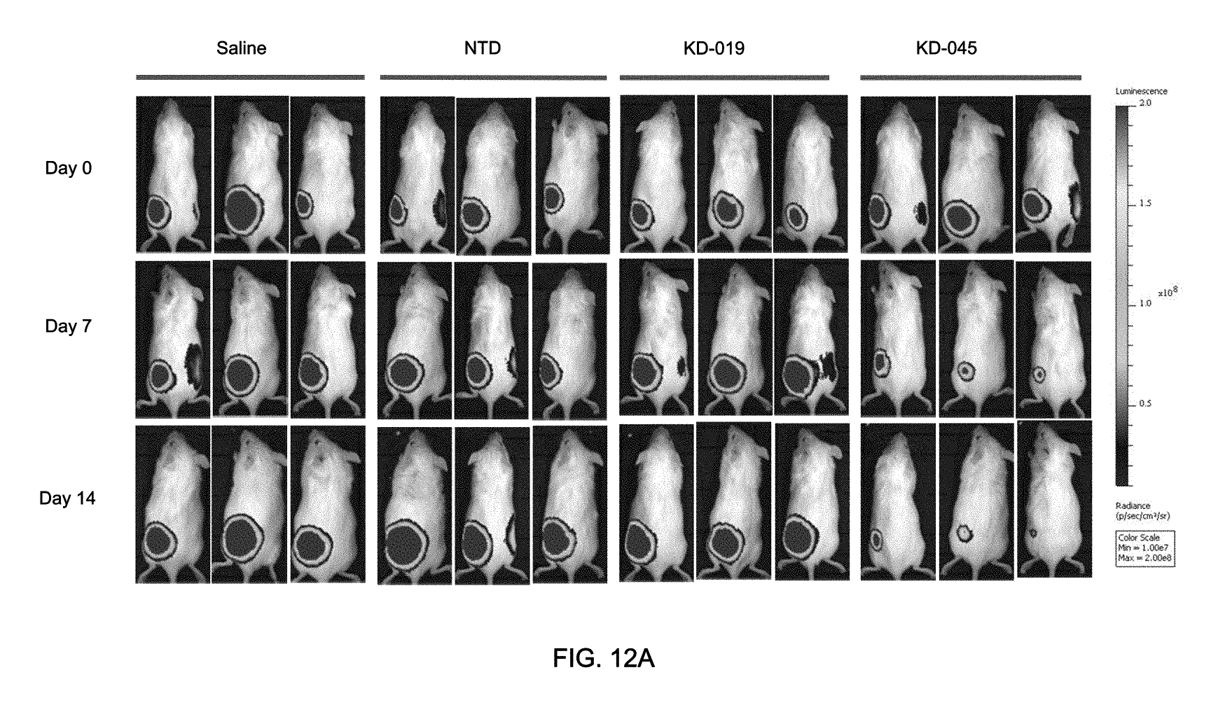

FIG. 11 shows the test results of tumor cell killing rate with colorectal cancer cell HCT116 as target cell. As can be seen from FIG. 11, the specific CAR-T cell KD-045 targeting to CD47 in embodiment 4 has a significant killing effect on the colorectal cancer cell HCT116 (obviously higher than the two control groups), and the tumor killing rate is over 50% at the ratio of effector to target 5:1.

The animal protocol was approved by the Institutional Review Board at Kunming Institute of Zoology, Chinese Academy of Sciences. NOD-Prkdcscid IL2rgtm1/Bcgen, NOD-SCID IL-2 receptor gamma null mice (B-NDG) were purchased form Jiangsu Biocytogen Co., Ltd (Nantong, China). Five to six week old B-NDG mice were bred under specific pathogen-free conditions. 1×10\hat{ }6 stable luciferase transfected U251 or SMMC7721 or U937 cells were suspended in PBS containing 2-˜30% Matrigel (BD Bioscience) and subcutaneously injected into the B-NDG mice. After 7 days, the mice were anaesthetized and imaged using IVIS system followed by the intraperitoneal injection of 150 mg/kg D-luiferin (BioVison). When the mean tumor bioluminescence reached about 5×10\hat{ }7 photons/second, mice was dealt with normal saline (NS) or 1×10\hat{ }7 none transduced T cells (NTD), KD-019 CAR or KD-045 CAR T cells by intravenous injection. The bioluminescent signals were measured every week. The data were quantified using Living Image software (Caliper Life Science).

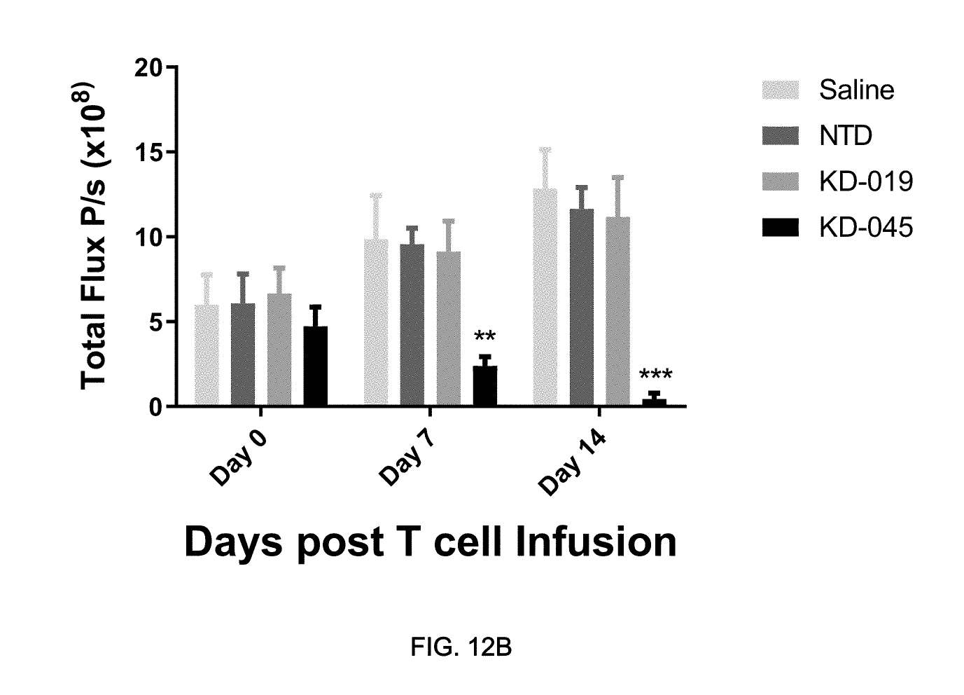

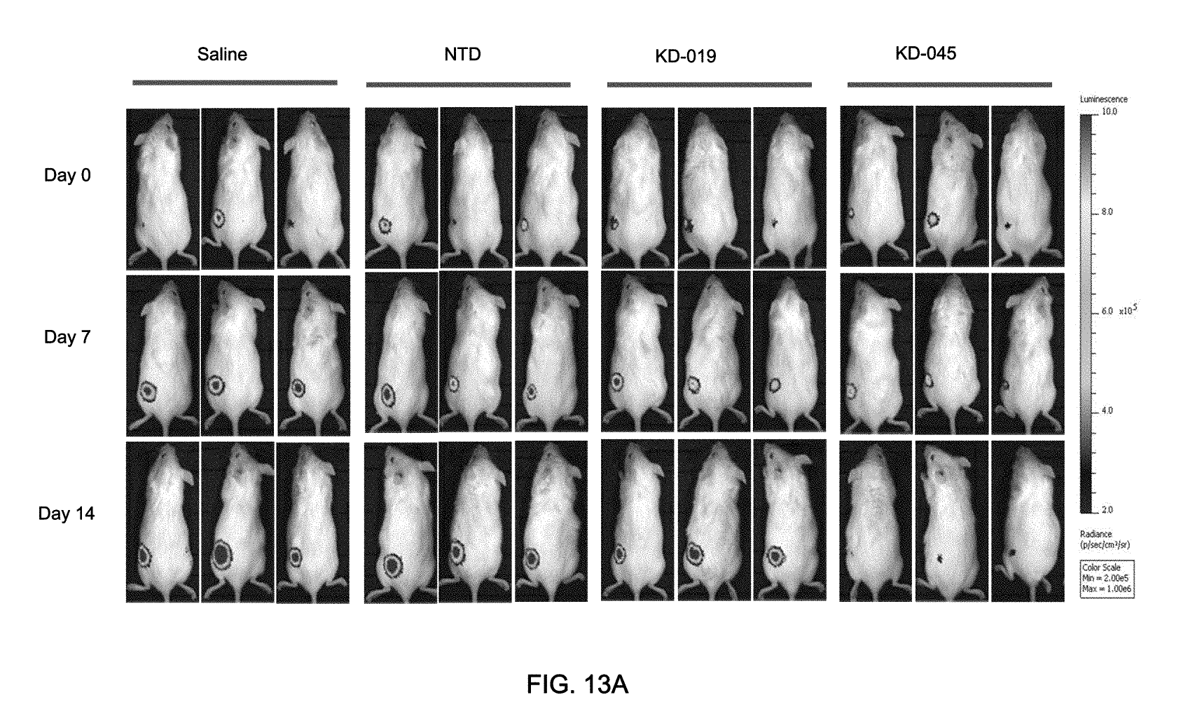

FIGS. 12A and 12B show the results of tumor suppressor test of CAR-T cells in NSG mice transplanted with glioblastoma U251 cells. From FIGS. 12A and 12B, we can see that the specific CAR-T cell KD-045 targeting to CD47 in embodiment 4 has a significant inhibitory effect on glioblastoma U251 xenografts. After inoculated tumor cells for 14 days, tumor growth is obviously suppressed (FIG. 12A), and the fluorescence intensity of the tumor was significantly lower than that of the three control groups (FIG. 12B).

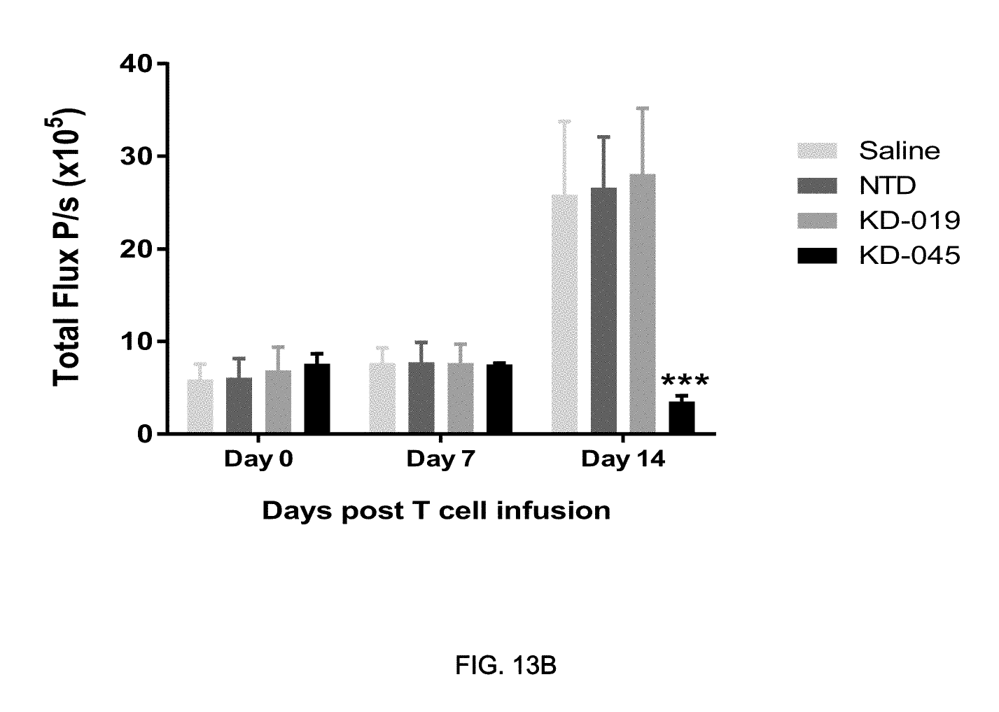

FIGS. 13A and 13B show the results of tumor suppressor test of CAR-T cells in NSG mice transplanted with medulloblastoma HTB186 cells. From FIGS. 13A and 13B, we can see that the specific CAR-T cell KD-045 targeting to CD47 in embodiment 4 has a significant inhibitory effect on medulloblastoma HTB186 xenografts. After inoculated tumor cells for 14 days, the tumor basically gone (FIG. 13A), and the fluorescence intensity of the tumor was significantly lower than that of the three control groups (FIG. 13B).

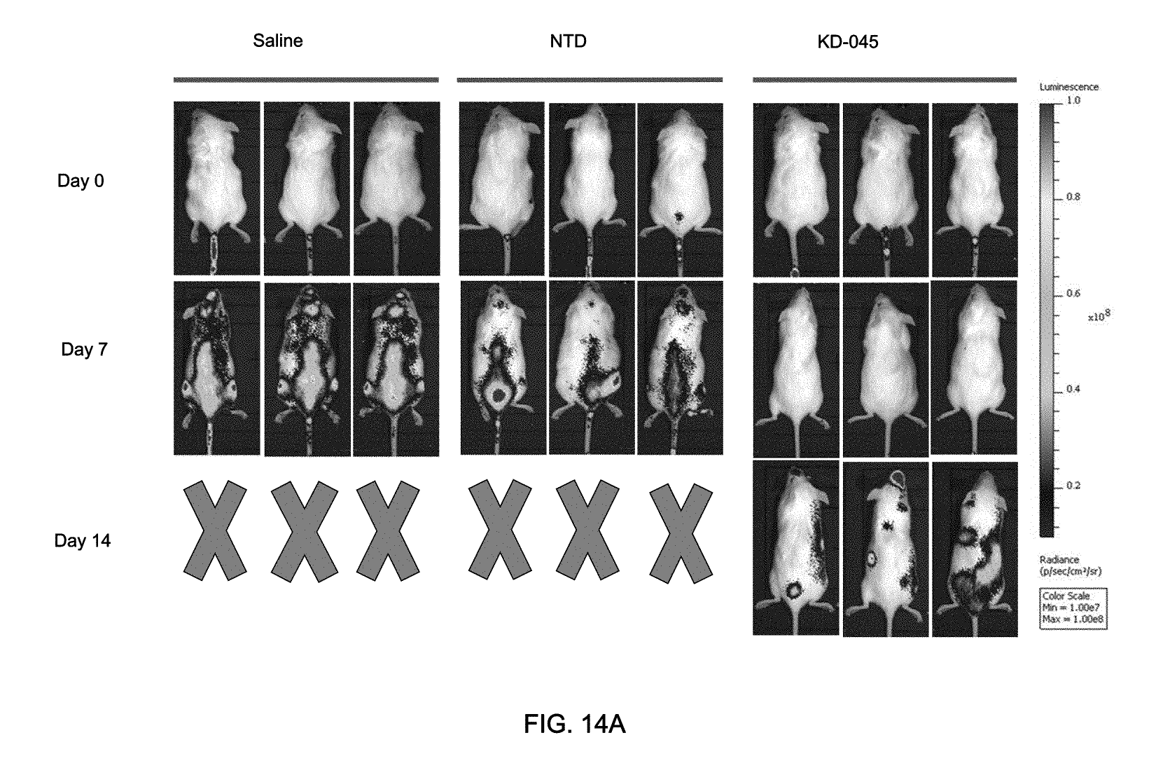

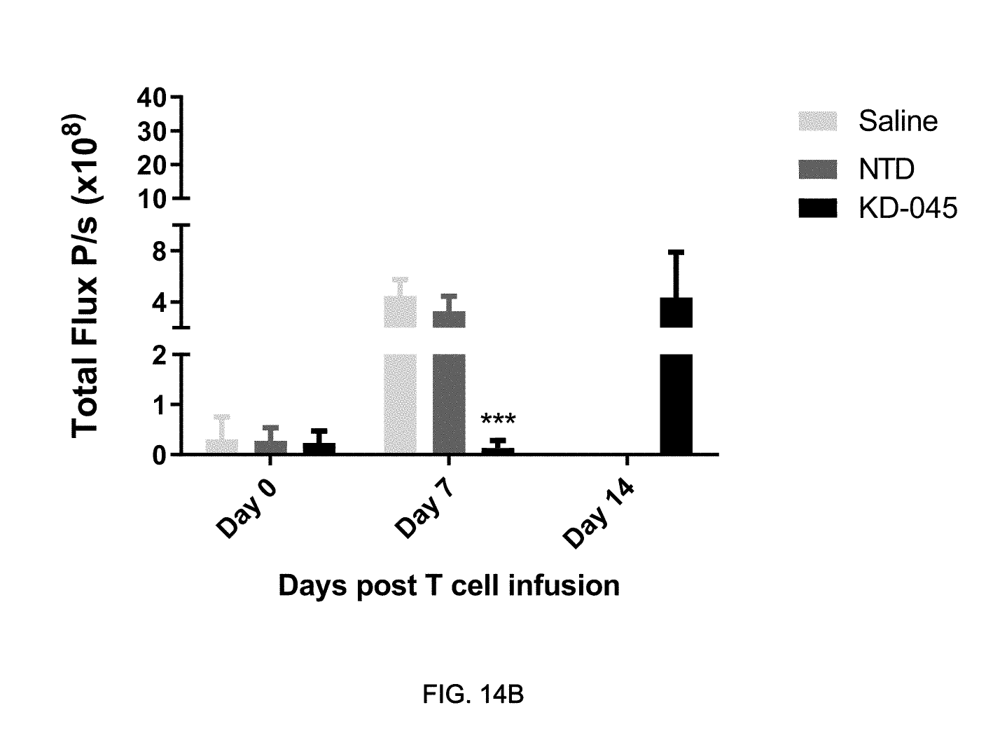

FIGS. 14A and 14B show the results of tumor suppressor test of CAR-T cells in NSG mice transplanted with U937 acute myeloid leukemia cells. From FIGS. 14A and 14B, we can see that the specific CAR-T cell KD-045 targeting to CD47 in embodiment 4 has a significant inhibitory effect on acute myeloid leukemia U937 xenografts. After inoculated tumor cells for 14 days, tumor growth is gone (FIG. 14A), and the fluorescence intensity of the tumor was lower than that of the three control groups (FIG. 14B).

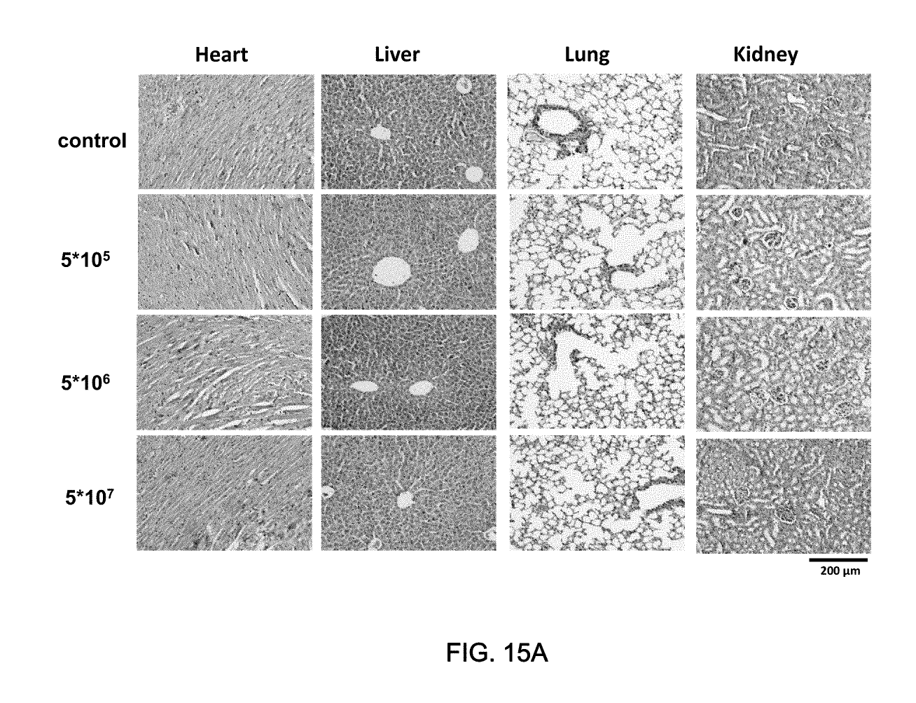

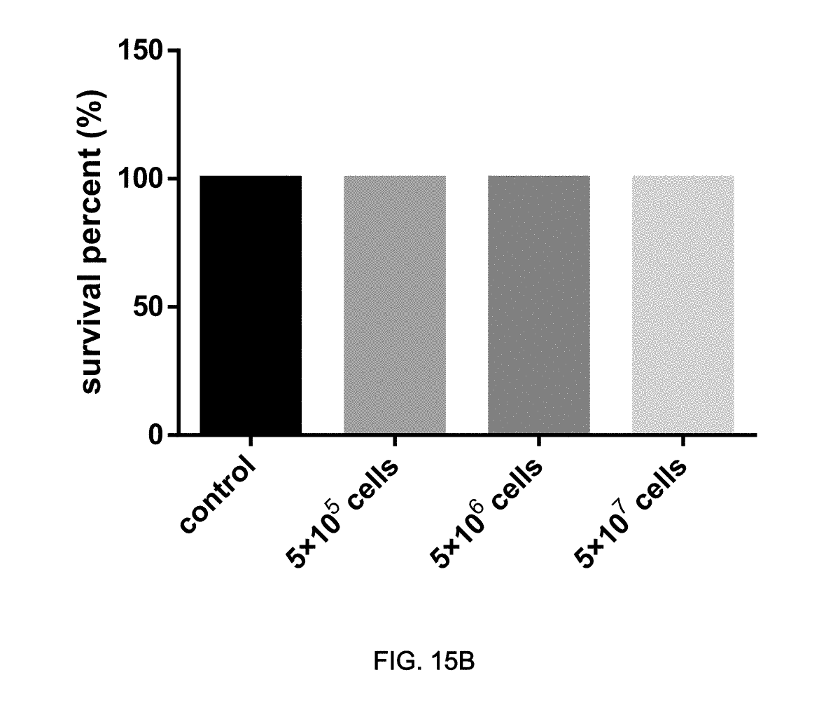

FIGS. 15A and 15B show the effects of different doses of KD-045 CAR-T cells on the main organs and survival term of mice. As can be seen from FIGS. 15A and 15B, the specific CAR-T cell KD-045 targeting to CD47 in example 4 does not cause inflammation, edema and necrosis in the main organs of the mice, such as heart, liver, lung, and kidney (FIG. 15A), and there is no negative effect on the survival term of mice (FIG. 15B).

The harvested samples of supernatant, obtained from the 16 hrs co-culture of effector cells with target cells at an E/T ratio of 5:1, were assessed for the levels of cytokine secretion. The concentrations of IFN-γ was measured using an enzyme-linked immunosorbent assay kit (BD Biosciences) in accordance with the operating manual.

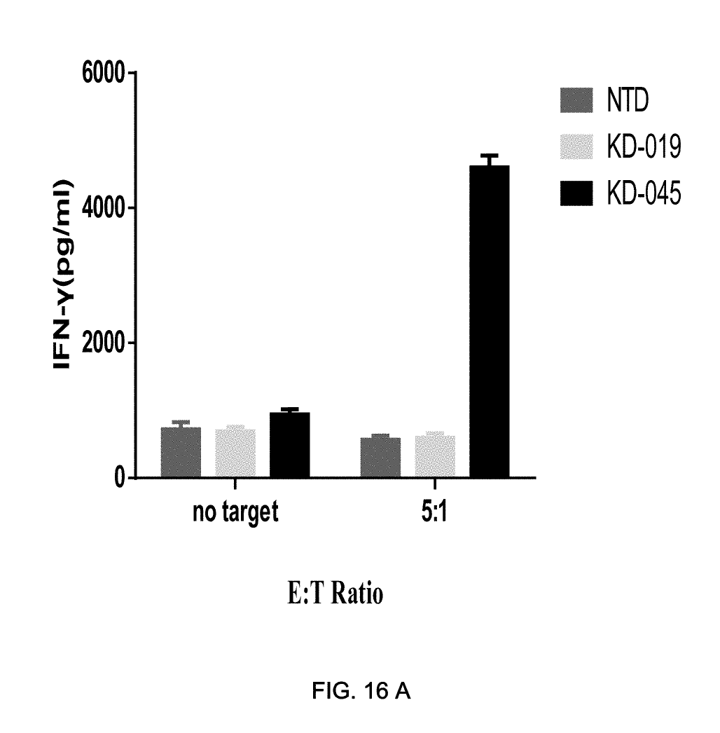

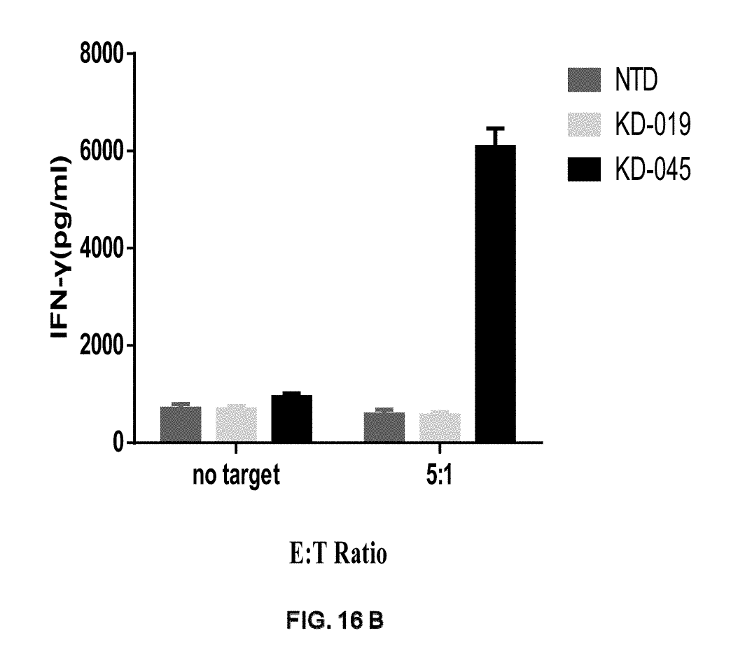

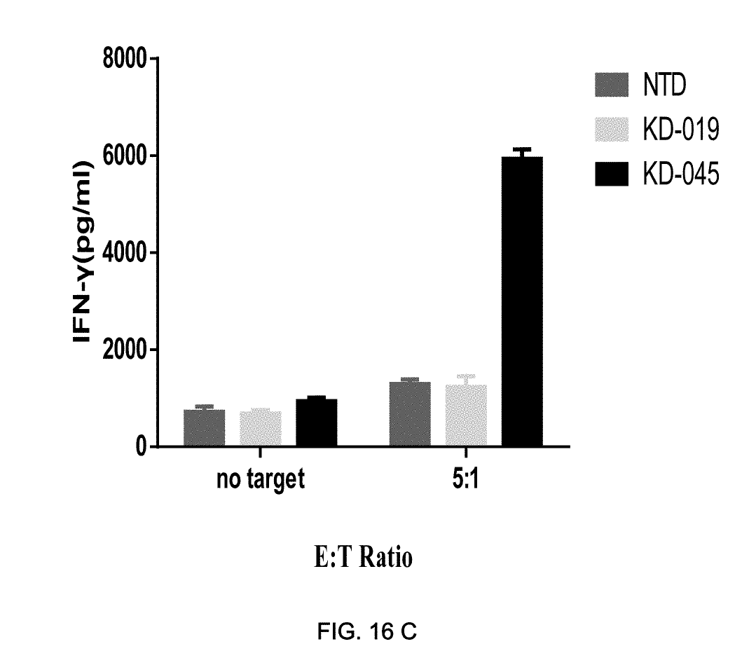

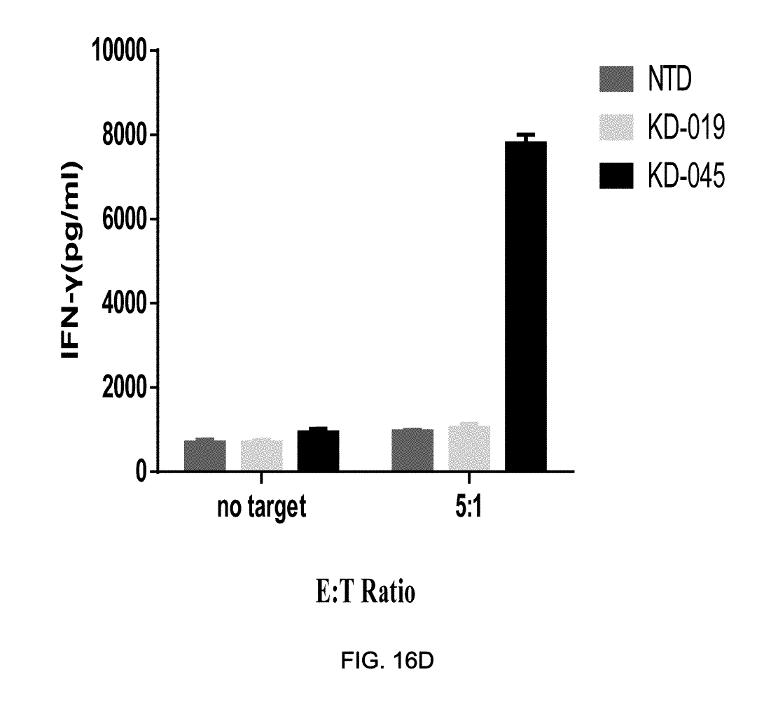

FIGS. 16A, 16B, 16C and 16D show the cytokine release of KD-045 CAR-T in vitro. As can be seen from FIGS. 16A, 16B, 16C and 16D, there show the cytokine IFN-γ release of KD-045 targeting to tumor cell lines: glioblastoma U251 cells (FIG. 16A), medulloblastoma HTB186 cell (FIG. 16B), acute myeloid leukemia U937 cells (FIG. 16C) and liver cancer SMMC7721 cells (FIG. 16D).

From the results of the above FIGS. 6 to 14B, it can be seen that the specific CAR-T cells of the target CD47 of the invention can specifically identify the CD47 positive tumor cells with a target killing capability.

Therefore, the specific chimeric antigen receptor targeting to CD47 and the specific CAR-T cells targeting to CD47 infected by its viral vector can be applied to the treatment of liver cancer, ovarian cancer, gastric cancer, lung cancer, prostate cancer, breast cancer, neuroglioma or neuroblastoma, pancreatic, bladder, and colon cancer, renal cell carcinoma, leukemia, lymphoma, multiple myeloma, melanoma, and their combinations.

It can be understood that although the instruction is described in the embodiment, not each embodiment comprises only one independent technical proposal. This description of the instruction is only to be clear, and the skilled in this field should take as a whole, and the technical solutions in embodiments can also be suitably combined to forms other embodiments can be understood by technicians in the field.

A series of detailed descriptions listed are only specific instructions for the embodiment of the invention. They are not used to limit the scope of protection of the invention. The equivalent embodiment methods or changes that are not separated from the topic of the invention should be comprised in the scope of the protection of the invention.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A protein targeting a human CD47, wherein the protein is a human SIRPα protein or a human SIRPα protein functional variant; and wherein the human SIRPα protein comprises amino acid sequences of SEQ ID NO:4, SEQ ID NO:5, SEQ ID NO:6, SEQ ID NO:7, SEQ ID NO:8 or SEQ ID NO:9, and the human SIRPα protein functional variant comprises amino acid sequence having 70%˜99% identity to one of the amino acid sequences of SEQ ID NO:4, SEQ ID NO:5, SEQ ID NO:6, SEQ ID NO:7, SEQ ID NO:8 or SEQ ID NO:9.

2. A chimeric antigen receptor targeting a human CD47, comprising:
an amino acid sequence from an amino terminal to a carboxyl terminal of a guiding sequence,
an extracellular domain targeting the human CD47,
a transmembrane domain and an intracellular signaling domain; wherein
immune response cells modified by a human SIRPα protein targeting the human CD47 have a killing efficiency of 50%˜90% at the ratio of effector to target 5:1, and an extracellular domain targeting the human CD47 comprises a human SIRPα protein or a human SIRPα protein functional variant; and wherein the human SIRPα protein comprises an amino acid sequence of SEQ ID NO:4, SEQ ID NO:5, SEQ ID NO:6, SEQ ID NO:7, SEQ ID NO:8 or SEQ ID NO:9, the human SIRPα protein functional variant comprises amino acid sequence having 70%˜99% identity to one of the amino acid sequences of SEQ ID NO:4, SEQ ID NO:5, SEQ ID NO:6, SEQ ID NO:7, SEQ ID NO:8 or SEQ ID NO:9, and a CD47 receptor of the human SIRPα protein targeting the human CD47; and
a hinge region.

3. The chimeric antigen receptor targeting the human CD47 according to claim 2, wherein
the amino acid sequence of the guiding sequence is the sequence of SEQ ID NO:3; and/or
the hinge region comprises a human CD8 having the amino acid sequence of SEQ ID NO:10; and/or
the transmembrane domain comprises a human CD8 polypeptide having the amino acid sequence of SEQ ID NO:11; and/or
the intracellular signaling domain comprises a human 4-1BB intracellular domain, a human CD28 intracellular domain, and a human CD3 zeta intracellular domain;
wherein an amino acid sequence of the human 4-1BB intracellular domain is the sequence of SEQ ID NO:12;
an amino acid sequence of the human CD28 intracellular domain is the sequence of SEQ ID NO:13; and
an amino acid sequence of the human CD3 zeta intracellular domain is the sequence of SEQ ID NO:14.

4. The chimeric antigen receptor targeting the human CD47 according to claim 2, wherein amino acid modifications of the human SIRPα protein comprise substitution, deletion and addition of amino acids of the human SIRPα protein, and amino acid modifications of homologous polypeptides of the human SIRPα protein comprise substitution, deletion and addition of amino acids of derived peptides derived from the human SIRPα protein.

5. The chimeric antigen receptor targeting the human CD47 according to claim 2, wherein an amino acid sequence from an amino terminal to a carboxyl terminal of the chimeric antigen receptor comprises the guiding sequence, a human SIRPα sequence selected from the group consisting of SEQ ID NO:4, SEQ ID NO:5, SEQ ID NO:6, SEQ ID NO:7, SEQ ID NO:8 and SEQ ID NO:9, a human CD8 hinge region sequence, a human CD8 transmembrane region sequence, a human 4-1BB intracellular domain sequence, and a CD3 zeta intracellular domain sequence; and wherein the guiding sequence, the human SIRPα sequence, the human CD8 hinge region sequence, the human CD8 transmembrane region sequence, the human 4-1BB intracellular domain sequence, and the CD3 zeta intracellular domain sequence are sequentially connected.

6. A nucleic acid molecule encoding the chimeric antigen receptor of claim 2, comprising: nucleotide sequences sequentially composed of a nucleotide sequence encoding the guiding sequence, a nucleotide sequence encoding the human SIRPα protein, a nucleotide sequence encoding the hinge region, a nucleotide sequence encoding the transmembrane domain, and a nucleotide sequence encoding the intracellular signaling domain from 5′ to 3′; and wherein the nucleotide sequence encoding the human SIRPα protein comprising the amino acid sequence of SEQ ID NO:4, SEQ ID NO:5, SEQ ID NO:6, SEQ ID NO:7, SEQ ID NO:8 or SEQ ID NO:9, the hinge region is a human CD8 hinge region, the transmembrane domain is a human CD8 transmembrane domain, the intracellular signaling domain comprises a human 4-1BB intracellular domain, and/or a human CD28 intracellular domain, and a CD3 zeta intracellular domain.

7. The nucleic acid molecule according to claim 6, wherein
the nucleotide sequence encoding the guiding sequence is the sequence of SEQ ID NO:16 or has 80%, 85%, 90%, 95% or 99% identity to the sequence of SEQ ID NO:16;
a nucleotide sequence encoding the human SIRPα targeting the human CD47 is the sequence selected from the group consisting of SEQ ID NO:17, SEQ ID NO:18, SEQ ID NO:19, SEQ ID NO:20, SEQ ID NO:21, and SEQ ID NO:22, or has 80%, 85%, 90%, 95% or 99% identity to the sequence selected from the group consisting of SEQ ID NO:17, SEQ ID NO:18, SEQ ID NO:19, SEQ ID NO:20, SEQ ID NO:21, and SEQ ID NO:22;
a nucleotide sequence encoding the human CD8 hinge region is the sequence of SEQ ID NO:23 or has 80%, 85%, 90%, 95% or 99% identity to the sequence of SEQ ID NO:23;
a nucleotide sequence encoding the human CD8 transmembrane region is the sequence of SEQ ID NO:24 or has 80%, 85%, 90%, 95% or 99% identity to the sequence of SEQ ID NO:24;
a nucleotide sequence encoding the human 4-1BB intracellular domain is the sequence of SEQ ID NO:25 or has 80%, 85%, 90%, 95% or 99% identity to the sequence of SEQ ID NO:25;
a nucleotide sequence encoding the CD28 intracellular domain is the sequence of SEQ ID NO:26 or has 80%, 85%, 90%, 95% or 99% identity to the sequence of SEQ ID NO:26;
a nucleotide sequence encoding the CD3 zeta intracellular domain is the sequence of SEQ ID NO:27 or SEQ ID NO:28 or has 80%, 85%, 90%, 95% or 99% identity to the sequence of SEQ ID NO:27

8. A promoter used to initiate an expression of the nucleic acid molecule encoding the chimeric antigen receptor of claim 6, wherein the promoter is an EF1 alpha promoter having the sequence of SEQ ID NO:29, or an EFS promoter having the sequence of SEQ ID NO:30.

9. CAR-T cells, comprising:
the chimeric antigen receptor targeting the human CD47 according to claim 2,
a first nucleic acid molecule comprising nucleotide sequences sequentially composed of the guiding sequence, a nucleotide sequence encoding the human SIRPα protein, a nucleotide sequence of encoding the transmembrane domain, a nucleotide sequence encoding the intracellular signaling domain from 5′ to 3; wherein the transmembrane domain comprises a human CD8 transmembrane domain, the intracellular domain comprises an immune receptor tyrosine activation sequence and a costimulatory signaling domain; and the first nucleic acid molecule further comprises a nucleotide sequence encoding the hinge region; or
a second nucleic acid molecule comprising nucleotide sequences sequentially composed of the guiding sequence, a nucleotide sequence encoding the human SIRPα protein, a nucleotide sequence of encoding the transmembrane domain, a nucleotide sequence encoding the intracellular signaling domain from 5′ to 3; wherein the transmembrane domain comprises a human CD8 transmembrane domain, the intracellular domain comprises an immune receptor tyrosine activation sequence and a costimulatory signaling domain.

10. The CAR-T cells according to claim 9, wherein the CAR-T cells are produced from immune response cells selected from the group consisting of T cells, natural killer cells, cytotoxic T lymphocytes, regulatory T cells, human embryonic stem cells or pluripotent stem cells; and wherein the human embryonic stem cells and the pluripotent stem cells have the potential to differentiate into lymphoid cells.

11. A pharmaceutical composition of the CAR-T cells according to claim 9 for treatment or prevention of tumor diseases, malaises or health disorder, comprising:
an effective dose of the CAR-T cells and an acceptable dose of excipient in pharmacy; and wherein the tumor diseases, malaises or health disorder are related to a specific interaction between the human SIRPα protein and a human SIRPα protein ligand CD47; and wherein the tumor disease, malaises or health disorder comprise pathogen infection, autoimmune disease, inflammatory disease, allograft, graft rejection and senescence.

12. A kit comprising the CAR-T cells according to claim 9 for treatment or prevention or diagnosis of tumor diseases, malaises or health disorder, wherein
the kit comprises an effective dose of the CAR-T cells and an acceptable dose of excipient in pharmacy; and wherein the tumor diseases, malaises, or health disorder are related to a specific interaction between the human SIRPα protein and a human SIRPα protein ligand human CD47.

13. A method for treatment or prevention of tumor diseases, health disorder, discomfort or senescence, comprising: using the nucleic acid molecule according to claim 6 in the treatment or prevention of the tumor diseases, health disorder, discomfort or senescence; wherein the treatment or the prevention of the tumor formation comprises:
reducing tumor burden in subjects, improving or prolonging survival of the subjects with tumor formation, or
increasing immune activation cytokines in response to tumor cells or pathogens of the subjects; wherein the tumor cells during the tumor formation expressing human CD47 proteins on surfaces of the tumor cells are selected from the group consisting of glioblastoma, medulloblastoma, acute myeloid leukemia, liver cancer, pancreatic cancer, colon cancer, ovarian cancer, gastric cancer, lung cancer, prostate cancer cell, breast cancer or neuroblastoma, bladder cancer, renal cell carcinoma, leukemia, lymphoma, multiple myeloma, and melanoma; wherein increasing immune activation cytokines in response to tumor cells or pathogens of the subjects is achieved by subjecting the CAR-T cells according to claim 10 to treat the tumor or the tumor formation by intravenous infusion, or intratumoral injection.


SEQUENCE LISTING (33 sequences)

SEQ ID NO: 1 - protein, 504 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 2 - DNA, 1515 bp, Homo sapiens
atggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtga

SEQ ID NO: 3 - protein, 21 aa, Artificial Sequence
  the amino acid sequence of the guided peptide      is synthesized
MALPVTALLLPLALLLHAARP

SEQ ID NO: 4 - protein, 248 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSE

SEQ ID NO: 5 - protein, 137 aa, Homo sapiens
ASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPA

SEQ ID NO: 6 - protein, 137 aa, Homo sapiens
EELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSF

SEQ ID NO: 7 - protein, 154 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVS

SEQ ID NO: 8 - protein, 116 aa, Homo sapiens
EELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAK

SEQ ID NO: 9 - protein, 504 aa, Homo sapiens; identical to SEQ ID NO: 1

SEQ ID NO: 10 - protein, 45 aa, Homo sapiens
TTTPAPRPPTPAPTIASQPLSLRPEACRPAAGGAVHTRGLDFACD

SEQ ID NO: 11 - protein, 24 aa, Homo sapiens
IYIWAPLAGTCGVLLLSLVITLYC

SEQ ID NO: 12 - protein, 42 aa, Homo sapiens
KRGRKKLLYIFKQPFMRPVQTTQEEDGCSCRFPEEEEGGCEL

SEQ ID NO: 13 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 14 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - DNA, 63 bp, Artificial Sequence
  the sequence is synthetized
atggccctgcccgtcaccgctctgctgctgccccttgctctgcttcttcatgcagcaaggccg

SEQ ID NO: 17 - DNA, 744 bp, Artificial Sequence
  the sequence is synthetized
atggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgag

SEQ ID NO: 18 - DNA, 411 bp, Artificial Sequence
  the sequence is synthetized
gcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcg

SEQ ID NO: 19 - DNA, 411 bp, Artificial Sequence
  the sequence is synthetized
gaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttc

SEQ ID NO: 20 - DNA, 462 bp, Artificial Sequence
  the sequence is synthetized
atggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcg

SEQ ID NO: 21 - DNA, 348 bp, Artificial Sequence
  the sequence is synthetized
gaagaattgcaagtaattcaaccagataaatcagttctggttgcagcgggggaaactgccaccctcagatgcactgcaacatctttgatacctgttggccctatccaatggtttcgaggagcaggaccaggccgcgaacttatatacaatcagaaggaggggcatttccctcgcgtgacaacggtctcagatctgaccaaacgcaacaatatggatttttccatacgcataggcaacataactcctgctgatgctggcacatattactgtgtcaaatttcggaagggctcaccggatgatgtcgagtttaagtccggggctggtacggagttgtccgtcagggcaaaa

SEQ ID NO: 22 - DNA, 1512 bp, Artificial Sequence
  the sequence is synthetized
atggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaag

SEQ ID NO: 23 - DNA, 135 bp, Artificial Sequence
  the sequence is synthetized
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 24 - DNA, 72 bp, Artificial Sequence
  the sequence is synthetized
atctacatctgggcgcccttggccgggacttgtggggtccttctcctgtcactggttatcaccctttactgc

SEQ ID NO: 25 - DNA, 126 bp, Artificial Sequence
  the sequence is synthetized
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 26 - DNA, 321 bp, Artificial Sequence
  the sequence is synthetized
attgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 27 - DNA, 336 bp, Artificial Sequence
  the sequence is synthetized
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 28 - DNA, 336 bp, Artificial Sequence
  the sequence is synthetized
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 29 - DNA, 1259 bp, Artificial Sequence
  the sequence is synthetized
tgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 30 - DNA, 256 bp, Artificial Sequence
  the sequence is synthetized
taggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 31 - DNA, 63 bp, Artificial Sequence
  the sequence is synthetized
cactttggcgccggctcgagggggcccgggtgcaaagatggataaagttttaaacagagagga

SEQ ID NO: 32 - DNA, 60 bp, Artificial Sequence
  the sequence is synthetized
cactttggcgccggctcgagggggcccgggtaggtcttgaaaggagtgggaattggctcc

SEQ ID NO: 33 - DNA, 59 bp, Artificial Sequence
  the sequence is synthetized
tccagaggttgattgtcgacttaacgcgtttagcgagggggcagggcctgcatgtgaag